SEQUENCE LISTING (7 sequences)

SEQ ID NO: 1 - protein, 39 aa, Homo sapiens
KQFTKAELSQLLKDIDGYGGIALPELIATMFHTSGYDTQ

SEQ ID NO: 2 - protein, 23 aa, Saccharomyces cerevisiae
MAGWDIFGWFRDVLASLGLWNKH

SEQ ID NO: 3 - protein, 15 aa, Homo sapiens
KQFTKAELSQLLKDI

SEQ ID NO: 4 - protein, 15 aa, Saccharomyces cerevisiae
MAGWDIFGWFRDVLA

SEQ ID NO: 5 - protein, 123 aa, Homo sapiens
KQFTKCELSQLLKDIDGYGGIALPELICTMFHTSGYDTQAIVENNESTEYGLFQISNKLWCKSSQVPQSRNICDISCDKFLDDDITDDIMCAKKILDIKGIDYWLAHKALCTEKLEQWLCEKL

SEQ ID NO: 6 - protein, 123 aa, Homo sapiens
KQFTKAELSQLLKDIDGYGGIALPELIATMFHTSGYDTQAIVENNESTEYGLFQISNKLWAKSSQVPQSRNIADISADKFLDDDITDDIMAAKKILDIKGIDYWLAHKALATEKLEQWLAEKL

SEQ ID NO: 7 - protein, 190 aa, Saccharomyces cerevisiae
MAGWDIFGWFRDVLASLGLWNKHGKLLFLGLDNAGKTTLLHMLKNDRLATLQPTWHPTSEELAIGNIKFTTFDLGGHIQARRLWKDYFPEVNGIVFLVDAADPERFDEARVELDALFNIAELKDVPFVILGNKIDAPNAVSEAELRSALGLLNTTGSQRIEGQRPVEVFMCSVVMRNGYLEAFQWLSQYI

(19) United States
(12) Patent Application Publication
(10) Pub. No.: US 2022/0323391 A1
(43) Pub. Date: Oct. 13, 2022

(54) COMBINATION OF A CHEMOTHERAPEUTIC AGENT AND ALPHA-LACTOGLUBULIN-OLEIC ACID COMPLEX FOR CANCER THERAPY

(71) Applicant: HAMLET PHARMA AB, Lund (SE)

(72) Inventors: Tran Thi HIEN, Lund (SE); Catharina SVANBORG, Malmo (SE)

(21) Appl. No.: 17/636,728

(22) PCT Filed: Aug. 19, 2020
(86) PCT No.: PCT/EP2020/073261

§ 371 Date: Feb. 18, 2022

(30) Foreign Application Priority Data
Aug. 20, 2019 (GB) 1911948.6
Mar. 19, 2020 (GB) 2004013.5

(51) Int. Cl. A61K 31/201; A61K 31/704; A61K 38/38; A61P 35/00; A61K 47/54

(52) U.S. Cl. CPC A61K 31/201; A61K 31/704; A61K 38/38; A61P 35/00; A61K 47/542

(57) ABSTRACT

A first chemotherapeutic agent and a second chemotherapeutic agent for use in cancer therapy, wherein the second chemotherapeutic agent comprises a biologically active complex having anti-tumour activity, wherein the biologically active complex consists of: a peptide of at least 10 amino acids comprising an alpha-helical structure; and oleic acid or an oleate salt, in a ratio of at least 3 oleic acid or oleate salt molecules per peptide molecule. Either the first or second chemotherapeutic agent for use in cancer therapy, for use in conjunction with the other chemotherapeutic agent, pharmaceutical compositions related thereto, and method of treatment.

### TECHNICAL FIELD

This invention relates to chemotherapeutic agents for use in combination chemotherapy in cancer therapy. The combination comprises a first chemotherapeutic agent and a second chemotherapeutic agent, wherein the second chemotherapeutic agent comprises a biologically active complex having anti-tumour activity, wherein the biologically active complex consists of: a peptide of at least 10 amino acids comprising an alpha-helical structure; and oleic acid or an oleate salt, in a ratio of at least 3 oleic acid or oleate salt molecules per peptide molecule. The invention also relates to pharmaceutical compositions and methods related thereto.

### BACKGROUND

Cancers form a highly heterogeneous, vastly complicated group of diseases. Yet, traits shared by many different cancer types have been elegantly defined, including the ability to outgrow and destroy healthy tissue and upset essential physiological functions. Targeted cancer therapies with improved tumor specificity are being developed, but when nothing else works, physicians resort to the use of radiation, surgery or highly toxic substances that kill cancer cells and healthy cells alike. Patients experience severe side effects because the drugs are not selective enough and “while you treat the cancer, the patient dies”.

Bladder cancer is common and the costliest cancer form in the USA, due to its high recurrence rate and a lack of curative therapies. With a prevalence of approximately 3.4 million, 430,000 new cases are diagnosed each year and about 200,000 deaths are estimated to occur annually. Survival depends on the recurrence rate and the risk for de-differentiation and invasive tumors may require cystectomy and systemic chemotherapy. Superficial papillary tumors, in contrast, are restricted to the mucosa and the short-term prognosis is excellent. Topical treatments such as Bacille Calmette-Guerin (BCG), Mitomycin, Thiotepa or Epirubicin have offered many patients long disease-free periods but may cause severe side effects (Malmström, P. U., Expert Rev Anticancer Ther, 4, 1057-1067 (2004); Schenkman, E. & Lamm, D., SciWorld J 4, 387-399 (2004)).

In more detail, the limitations of current bladder cancer therapies are reflected by a high recurrence rate in this patient group and a risk for progression to more malignant, invasive disease. The current standard treatment for non-muscle invasive bladder cancer (NMIBC) is transurethral resection of the bladder tumor. Intravesical chemotherapy is generally used as adjuvant therapy after resection and has been shown to reduce recurrence, but not progression of disease for patients with low-risk NMIBC (Kang, M et al., Oncotarget 7, 45479 (2016); Sylvester, R. J., et al., Eur Urol 69, 231-244 (2016)). Intravesical immunotherapy with bacillus Calmette-Guérin (BCG) is recommended after surgery and is superior to intravesical chemotherapy for preventing tumor recurrence (Kamat, A. M., et al. Lancet 388, 2796-2810 (2016)). Mitomycin C, which is widely used for intravesical chemotherapy of newly diagnosed superficial bladder cancer, reduces tumor recurrences and prolongs disease free interval (Wilhelm, S., et al., Nat Rev Mater 1, 16014 (2016)). These therapies are accompanied by significant side effects and a significant risk of tumor recurrence. There is a great, unmet need, for less toxic, more specific therapies with enhanced tumor-killing properties.

The present study provides a combination chemotherapy that shows a significant therapeutic effect against cancer, particularly bladder cancer.

### SUMMARY OF THE INVENTION

The invention relates to the use of two chemotherapeutic agents for the treatment of cancer. The agents are generally for use in a combination therapy, that is to say, they are both to be administered to the same subject over the course of a treatment. They may be for separate administration, or in the same composition. The agents may be for substantially contemporaneous administration, i.e. at the same time or during the same treatment session. Or, they may be for administration at separate times, but within the same treatment course.

In particular, the invention relates to a biologically active complex having anti-tumour activity, or an agent comprising such a complex, wherein the biologically active complex consists of: a peptide of at least 10 amino acids comprising an alpha-helical structure; and oleic acid or an oleate salt, in a ratio of at least 3 oleic acid or oleate salt molecules per peptide molecule, and wherein the chemotherapeutic agent is for use with another chemotherapeutic agent.

The inventors have identified that the combined effect of two chemotherapeutic agents provides unexpectedly high antitumour activity. Without being bound by theory, the inventors believe that when a biologically active complex according to the invention and another chemotherapeutic agent are administered in conjunction, the mechanism of action of the biologically active complexes upon tumor cells acts to potentiate the chemotherapeutic effect of the other chemotherapeutic agent as compared with the effect of that chemotherapeutic agent when used in isolation. Again, without wishing to be bound by theory, it is further believed that the biologically active complexes according to the invention can disrupt tumor membrane integrity and increase uptake of co-administered chemotherapeutic agents into the tumor cell and/or the tumor cell nucleus, and/or reduce the tumor cell's ability to withstand downstream effects of other chemotherapeutic agents, potentially through effects of the biologically active complex on expression of molecules involved in the cancer pathway. A further important effect of the combination is increased precision of targeting tumour cells over healthy cells.

In one aspect, the invention provides a first chemotherapeutic agent and a second chemotherapeutic agent for use in cancer therapy, wherein the second chemotherapeutic agent comprises a biologically active complex having anti-tumour activity, wherein the biologically active complex consists of: a peptide of at least 10 amino acids comprising an alpha-helical structure; and oleic acid or an oleate salt, in a ratio of at least 3 oleic acid or oleate salt molecules per peptide molecule.

According to another aspect, the invention provides a first chemotherapeutic agent for use in cancer therapy, which is to be used in conjunction with a second chemotherapeutic agent comprising a biologically active complex having anti-tumour activity, wherein the biologically active complex consists of: a peptide of at least 10 amino acids comprising an alpha-helical structure; and oleic acid or an oleate salt, in a ratio of at least 3 oleic acid or oleate salt molecules per peptide molecule.

In this, and optionally in other aspects, the first chemotherapeutic agent is for use in cancer therapy to be administered to a subject, wherein the subject to which the agent is to be administered has received, or is in the process of receiving, or is going to receive a dose or course of treatment with the second chemotherapeutic agent.

According to a further aspect, the invention provides a second chemotherapeutic agent for use in cancer therapy, which is to be used in conjunction with a first chemotherapeutic agent, wherein the second chemotherapeutic agent comprises a biologically active complex having anti-tumour activity, wherein the biologically active complex consists of: a peptide of at least 10 amino acids comprising an alpha-helical structure; and oleic acid or an oleate salt, in a ratio of at least 3 oleic acid or oleate salt molecules per peptide molecule.

In this, and optionally in other aspects, the second chemotherapeutic agent is for use in cancer therapy to be administered to a subject, wherein the subject to which the agent is to be administered has received, or is in the process of receiving, or is going to receive a dose or course of treatment with the first chemotherapeutic agent.

By chemotherapeutic agent, we are referring to agents that are used in cancer therapy, i.e. in treatment and/or prevention of cancer. Prevention can include prevention of cancer occurring in the first place as well as prevention of cancer recurring. By referring to a first chemotherapeutic agent and a second chemotherapeutic agent, we mean that the chemotherapeutic agents are different from each other. The first chemotherapeutic agent can be, for example, an intravesical chemotherapeutic agent, a topical chemotherapeutic agent, and/or a DNA-interactive chemotherapeutic agent. DNA-interactive chemotherapeutic agents include DNA-alkylator, DNA-crosslinker and DNA-intercalator chemotherapeutic agents.

The agents may be for combined administration, for example, as part of the same composition. While it is possible that the first chemotherapeutic agent and second chemotherapeutic agent can be chemically bound to each other, for example by one or more covalent bonds, typically the first chemotherapeutic agent and second chemotherapeutic agent are not chemically bound to each other.

In one embodiment, the first chemotherapeutic agent is an intravesical chemotherapeutic agent. By intravesical chemotherapeutic agent, we are referring to a chemotherapeutic agent that is or can be used in treatment of bladder cancer. Intravesical chemotherapeutic agents include atezolizumab, avelumab, Bacille Calmette-Guerin, bevacizumab, carbozantinib, cephalexin, ciprofloxacin, cisplatin, doxorubicin hydrochloride, durvalumab, eflornithine, epirubicin, erdafitinib, erlotinib, fenretinide, gemcitabine, gefitinib, lapatinib, mitomycin C, nivolumab, pazobanib, pembrolizumab, rapamycin, selenium, sorafenib, thiotepa, urocidin, valrubicin, and vicinium. In a preferred embodiment, the first chemotherapeutic agent is selected from the group consisting of: thiotepa, mitomycin C, Bacille Calmette-Guerin, or epirubicin, and is preferably mitomycin C or epirubicin. In one embodiment it is mitomycin C. In another embodiment it is epirubicin. Mitomycin C can also be administered in the form of a prodrug, such as apaziquone. The mitomycins are a family of natural products, which includes mitomycin A, mitomycin B and mitomycin C. Of the mitomycins, it is typically mitomycin C that is used in chemotherapy. Generally, the term “mitomycin” as used herein is a shorthand for mitomycin C, particularly in the figures and examples, unless it is clear the term is being used to refer to the family of mitomycin compounds.

The second chemotherapeutic agent comprises a biologically active complex having anti-tumour activity. In one embodiment, the second chemotherapeutic agent consists of a biologically active complex having anti-tumour activity. By “anti-tumour activity” we are referring to the ability of the complex to kill, weaken, slow the growth, or exhibit any other deleterious effect on a tumour cell. Anti-tumour activity can be readily screened for by any of a number of well-established techniques, including the techniques discussed in further detail below. Preferably, the complex is a non-covalent complex of the peptide and oleic acid or oleate salt molecules.

The biologically active complex consists of: a peptide of at least 10 amino acids comprising an alpha-helical structure; and oleic acid or an oleate salt, in a ratio of at least 3 oleic acid or oleate salt molecules per peptide molecule. A number of such biologically active complexes have been previously identified (see, for example, WO 2010/079362, WO 2012/052310, WO 2018/116165 and PCT/EP2019/066409). The peptide can be obtained from any suitable source, such as expression from a natural host (e.g. yeast, human, non-human primate, cow, sheep, goat, horse and the like), recombinant expression or chemical synthesis. The peptide can comprise or have the sequence of, or comprise or be a fragment and/or variant sequence of, the wild-type peptide sequence of a peptide from a yeast, human, non-human primate, cow, sheep, goat, horse and the like. In one embodiment the peptide sequence is of eukaryotic origin, preferably mammalian or yeast origin. Preferably the peptide of mammalian origin is a human sequence or a fragment and/or variant of a human sequence. Preferably the peptide of yeast origin is a Saccharomyces cerevisiae sequence or a fragment and/or variant of a Saccharomyces cerevisiae sequence.

In one embodiment, the peptide of the biologically active complex is of a protein which has membrane perturbing activity (see, for example, WO 2018/116165).

The peptide of the biologically active complex, where present in the complex, can have an increased conformational fluidity of three-dimensional structure as compared to the peptide alone as indicated by an increased peak width on at least some 1H NMR peaks of the complex as compared to the corresponding width of the peaks of a 1H NMR of the non-complexed peptide. The significance of this increase in conformational fluidity on anti-tumour activity of the complex has been discussed elsewhere (see, for example, PCT/EP2019/066409). In summary, the inventors have identified that peptides with diverse sequences can form complexes with oleate that share certain structural properties which result in the anti-tumour properties. These include having an alpha-helical structure, the ability to bind oleic acid or oleate, and having an increased conformational fluidity of three-dimensional structure upon binding. A marker of increased fluidity of three-dimensional structure is an increased peak width on at least some 1H NMR peaks of the complex as compared to the corresponding width of the peaks of a 1H NMR of the non-complexed peptide (i.e. when not in a complex with oleate). In one embodiment, there is an increased peak width on at least one 1H NMR peak of the complex as compared to the corresponding width of the peak of a 1H NMR of the non-complexed peptide. In one embodiment, the peptide has at least one tryptophan residue and the 1H NMR peak of at least one tryptophan indole proton in the complex is increased as compared to the corresponding width of the peak of a 1H NMR of the peptide alone. Another indicator of increased fluidity is a reduced chemical shift dispersion of 1H NMR peaks in a 1H NMR of the complex as compared to the corresponding peaks of a 1H NMR of the peptide alone.

The peptide of the biologically active complex can lack cysteine residues. Cysteine residues have the ability to form disulphide bonds, which typically brings rigidity to the three-dimensional tertiary structure of a peptide. Having a peptide without cysteine residues prevents this disulphide bond formation, which helps to increase the conformational fluidity of the peptide and enhance the formation and anti-tumour effect of the biologically active complex.

The peptide of the biologically active complex can be a fragment of a longer protein, so long as the peptide fragment exhibits anti-tumour activity in the biologically active complex. In an embodiment, the peptide of the biologically active complex is 50 amino acids or fewer in length, i.e. it consists of a sequence having up to 50 amino acids in length. In particular, it may be fewer than 45, 40, or 35 amino acids in length. It is preferably at least 10, 12, 15, 20 or 25 amino acids in length. While early studies used full length proteins, recent studies have shown that anti-tumour efficacy can be achieved with the use of suitable protein fragments or variants thereof. The shorter amino acid chain of such fragments allows for a simpler, faster and more reproducible manufacturing process, which is particularly important when considering manufacturing processes that must be compliant with Good Manufacturing Practices (GMP). As an example, such peptides can be efficiently synthesised chemically.

In a preferred embodiment, the peptide of the biologically active complex is alpha-lactalbumin or SAR-1, or a variant or fragment thereof, preferably an N-terminal fragment thereof. By N-terminal fragment, we are generally referring to the fragment comprising the N-terminal amino acid (or amino acid variant of the N-terminal amino acid) of the wild-type protein.

Biologically active complexes wherein the peptide is alpha-lactalbumin have been described previously, for example in the form of HAMLET (Human Alpha-lactalbumin Made LEthal to Tumor cells) and a number of peptides derived from alpha-lactalbumin have also been found to have therapeutic effects in their own right (see for example WO2012/069836). Examples of full-length alpha-lactalbumin sequences are SEQ ID Nos: 5 and 6.

(SEQ ID NO 5)
KQFTKCELSQ LLKDIDGYGG IALPELICTM FHTSGYDTQA
IVENNESTEY GLFQISNKLW CKSSQVPQSR NICDISCDKF
LDDDITDDIM CAKKILDIKG IDYWLAHKAL CTEKLEQWLC EKL
(SEQ ID NO 6)
KQFTKAELSQ LLKDIDGYGG IALPELIATM FHTSGYDTQA
IVENNESTEY GLFQISNKLW AKSSQVPQSR NIADISADKF
LDDDITDDIM AAKKILDIKG IDYWLAHKAL ATEKLEQWLA EKL

Biologically active complexes wherein the peptide is SAR1 or a fragment thereof have also been described previously (see for example WO2018/116165 and PCT/EP2019/066409). Preferably, the SAR1 is derived from yeast, or is a variant or fragment of a yeast sequence. An example of a full-length SAR1 sequence is SEQ ID No: 7.

(SEQ ID NO 7)
MAGWDIFGWF RDVLASLGLW NKHGKLLFLG LDNAGKTTLL
HMLKNDRLAT LQPTWHPTSE ELAIGNIKFT TFDLGGHIQA
RRLWKDYFPE VNGIVFLVDA ADPERFDEAR VELDALFNIA
ELKDVPFVIL GNKIDAPNAV SEAELRSALG LLNTTGSQRI
EGQRPVEVFM CSVVMRNGYL EAFQWLSQYI

The expression “variant” refers to proteins or polypeptides having a similar biological function but in which the amino acid sequence differs from the base sequence from which it is derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type.

By “conservative substitution” is meant the substitution of an amino acid by another amino acid of the same class, in which the classes are defined as follows:

### Class Amino Acid Examples

Nonpolar: A, V, L, I, P, M, F, W

Uncharged polar: G, S, T, C, Y, N, Q

Acidic: D, E

Basic: K, R, H.

As is well known to those skilled in the art, altering the primary structure of a peptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptide's conformation.

Non-conservative substitutions are possible provided that these do not interrupt the function of the peptide. Broadly speaking, fewer non-conservative substitutions will be possible without altering the biological activity of the polypeptides.

Determination of the effect of any substitution (and, indeed, of any amino acid deletion or insertion) is wholly within the routine capabilities of the skilled person, who can readily determine whether a variant polypeptide retains the fundamental properties and activity of the basic protein. For example, when determining whether a variant of the polypeptide falls within the scope of the invention, the skilled person will determine whether complexes comprising the variant retain biological activity (e.g tumour cell death) of complexes formed with unfolded forms of the native protein and the polypeptide has at least 60%, preferably at least 70%, more preferably at least 80%, yet more preferably 90%, 95%, 96%, 97%, 98%, 99% or 100% activity of the native protein.

Variants of the polypeptide may comprise or consist essentially of an amino acid sequence with at least 70% identity, for example at least 75%, 80%, 85%, 90%, 91%, 92%, 93%, 94%, 96%, 97%, 98% or 99% identity to a native protein sequence such as an alpha-lactalbumin or SAR1 sequence or to an active fragment of such a native protein sequence.

The level of sequence identity is suitably determined using the BLASTP computer program with the native protein sequences as the base sequence. This means that native protein sequences form the sequence against which the percentage identity is determined. The BLAST software is publicly available at http://blast.ncbi.nlm.nih.gov/Blast.cgi (accessible on 12 Mar. 2009).

In one embodiment, the variant is a variant that lacks cysteine residues.

Where the peptide is a fragment, suitably the peptide is up to 40 amino acids in length, for example up to 30 amino acids, or up to 25 amino acids in length. Typically, the peptide will be from 10-40 amino acids in length, for example, 11, 12, 13, 14, 15, 16, 17, 18, 19, 20, 21, 22, 23, 24, 25, 26, 27, 28, 29, 30, 31, 32, 33, 34, 35, 36, 37, 38 or 39 amino acids in length. In one embodiment it is 15 amino acids in length. In another, it is 23 amino acids in length and in another, 39 amino acids in length. In particular, the peptide can be an N-terminal fragment of a protein having an N-terminal alpha helical domain.

In a particularly preferred embodiment, the peptide of the biologically active complex comprises or consists of any of SEQ ID NOs: 1-4, or functional variants or fragments thereof, preferably SEQ ID NO: 1.

TABLE 1

| SEQ | | | | |
|---|---|---|---|---|
| ID | | | Parent | |
| NO | Sequence | Length | protein | ID |
| 1 | KQFTKAELSQLLKDIDGYGG | 39mer | α-lactalbumin | Alpha1H |
| | IALPELIATMFHTSGYDTQ | | | (α1H; α1O) |
| 2 | MAGWDIFGWFRDVLASLGLWNKH | 23mer | Sar1 | Sar1-alpha |
| 3 | KQFTKAELSQ LLKDI | 15mer | α-lactalbumin | Alpha1-P1 |
| 4 | MAGWDIFGWF RDVLA | 15mer | Sar1 | Sar1-alpha1 |

In a preferred embodiment, the peptide of the biologically active complex consists of SEQ ID NO: 1.

In one embodiment, the peptide of the biologically active complex comprises a truncated form of SEQ ID NO: 1 or SEQ ID NO: 2 of up to 19 amino acids in length.

In one embodiment, the first agent is mitomycin C and the second agent is SEQ ID NO. 1.

In one embodiment, the first agent is mitomycin C and the second agent is SEQ ID NO. 2.

In one embodiment, the first agent is mitomycin C and the second agent is SEQ ID NO. 3.

In one embodiment, the first agent is mitomycin C and the second agent is SEQ ID NO. 4.

In one embodiment, the first agent is epirubicin and the second agent is SEQ ID NO. 1.

In one embodiment, the first agent is epirubicin and the second agent is SEQ ID NO. 2.

In one embodiment, the first agent is epirubicin and the second agent is SEQ ID NO. 3.

In one embodiment, the first agent is epirubicin and the second agent is SEQ ID NO. 4.

Optional combinations of the chemotherapeutic agents are provided in table 2. The combinations shown in table 2 include, as the first chemotherapeutic agent, functional equivalents of the agents mentioned. The combinations further include, as the second chemotherapeutic agent, the peptides having the sequences shown in table 1. The possible combinations additionally comprise longer peptides comprising the sequences shown, or functional fragments or variants of peptides having the sequences shown.

TABLE 2

| | | | |
|---|---|---|---|
| Atezolizumab and | Atezolizumab and | Atezolizumab and | Atezolizumab and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Avelumab and | Avelumab and | Avelumab and | Avelumab and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Bacille Calmette- | Bacille Calmette- | Bacille Calmette- | Bacille Calmette- |
| Guerin and | Guerin and | Guerin and | Guerin and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Bevacizumab and | Bevacizumab and | Bevacizumab and | Bevacizumab and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Carbozantinib and | Carbozantinib and | Carbozantinib and | Carbozantinib and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Cephalexin and | Cephalexin and | Cephalexin and | Cephalexin and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Ciprofloxacin and | Ciprofloxacin and | Ciprofloxacin and | Ciprofloxacin and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Cisplatin and | Cisplatin and | Cisplatin and | Cisplatin and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Doxorubicin | Doxorubicin | Doxorubicin | Doxorubicin |
| hydrochloride and | hydrochloride and | hydrochloride and | hydrochloride and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Durvalumab and | Durvalumab and | Durvalumab and | Durvalumab and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Eflornithine and | Eflornithine and | Eflornithine and | Eflornithine and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Epirubicin and | Epirubicin and | Epirubicin and | Epirubicin and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Erdafitinib and | Erdafitinib and | Erdafitinib and | Erdafitinib and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Erlotinib and | Erlotinib and | Erlotinib and | Erlotinib and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Fenretinide and | Fenretinide and | Fenretinide and | Fenretinide and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Gemcitabine and | Gemcitabine and | Gemcitabine and | Gemcitabine and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Gefitinib and | Gefitinib and | Gefitinib and | Gefitinib and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Lapatinib and | Lapatinib and | Lapatinib and | Lapatinib and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Mitomycin C and | Mitomycin C and | Mitomycin C and | Mitomycin C and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Nivolumab and | Nivolumab and | Nivolumab and | Nivolumab and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Pazobanib and | Pazobanib and | Pazobanib and | Pazobanib and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Pembrolizumab and | Pembrolizumab and | Pembrolizumab | Pembrolizumab and |
| KQFTKAELSQ | MAGWDIFGWF | and KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Rapamycin and | Rapamycin and | Rapamycin and | Rapamycin and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Selenium and | Selenium and | Selenium and | Selenium and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Sorafenib and | Sorafenib and | Sorafenib and | Sorafenib and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Thiotepa and | Thiotepa and | Thiotepa and | Thiotepa and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Urocidin and | Urocidin and | Urocidin and | Urocidin and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Valrubicin and | Valrubicin and | Valrubicin and | Valrubicin and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |
| Vicinium and | Vicinium and | Vicinium and | Vicinium and |
| KQFTKAELSQ | MAGWDIFGWF | KQFTKAELSQ | MAGWDIFGWF |
| LLKDIDGYGG | RDVLASLGLW NKH | LLKDI | RDVLA |
| IALPELIATM | | | |
| FHTSGYDTQ | | | |

In one embodiment, the use is in treatment or prevention of carcinomas, lymphomas or brain tumours, preferably treatment or prevention of cancer of the GI tract, mucosal cancer, bladder cancer, kidney cancer, lung cancer, glioblastomas and skin papilloma, more preferably treatment or prevention of bladder cancer. Preferably, the cancer is a human cancer. In one embodiment it is the treatment of bladder cancer. In one embodiment, it is the prevention of bladder cancer.

The first chemotherapeutic agent may be administered or for administration in a dose of less than half the dose required to produce the therapeutic effect when it administered in the absence of the second chemotherapeutic agent. The first chemotherapeutic agent may be administered or for administration in an amount of at least 0.001, 0.01, 0.1, 0.5, 1, or 1.25 mg/kg of body weight, or at least 0.001, 0.01, 0.1, 0.5, 1, or 1.25 μg/kg of body weight. In certain embodiments, the first chemotherapeutic agent may be administered or for administration in an amount of at most 1000, 500, 100, 50, 10, 4, 2 and 1.5 mg/kg body weight, or at most 1000, 500, 50, 10, 4, 2 or 1.5 μg/kg body weight. In certain embodiments, the second chemotherapeutic agent may be administered or for administration in an amount of at least 0.001, 0.01, 0.1, 0.5, 1, 2, 4 or 8 mg/kg body weight, or at least 0.001, 0.01, 0.1, 1, 5, 10, 20, 40 or 45 μg/kg body weight. The second chemotherapeutic agent may be administered or for administration in an amount of at most 5000, 1000, 500, 100, 50, 25 or 10 mg/kg body weight, or at most 10000, 5000, 100, 500, 75 or 50 μg/kg body weight. Preferably the first chemotherapeutic agent is administered in a range between 0.5 and 2 mg/kg body weight and/or the second chemotherapeutic agent is administered in a range between 25 and 75 mg/kg body weight.

The first chemotherapeutic agent and second chemotherapeutic agent are generally administered in conjunction. The first chemotherapeutic agent and second chemotherapeutic agent can be administered in combination, simultaneously or sequentially. This may mean that they are administered as part of the same composition, in different compositions but at the same time, or as part of the same treatment programme or regime.

According to another aspect, the invention provides a pharmaceutical composition comprising a first chemotherapeutic agent, a second chemotherapeutic agent, and a pharmaceutically acceptable carrier, excipient and/or adjuvant, wherein the second chemotherapeutic agent comprises a biologically active complex having anti-tumour activity, wherein the biologically active complex consists of: a peptide of at least 10 amino acids comprising an alpha-helical structure; and oleic acid or an oleate salt, in a ratio of at least 3 oleic acid or oleate salt molecules per peptide molecule. The first chemotherapeutic agent and/or second chemotherapeutic agent of the pharmaceutical composition can further comprise any of the features described for any other aspects of the invention.

According to a further aspect, the invention provides a pharmaceutical composition according to the fourth aspect of the invention for use in therapy.

According to an additional aspect, the invention provides a method of treating or preventing cancer, the method comprising administration to a subject in need thereof of a first chemotherapeutic agent in conjunction with a second chemotherapeutic agent, wherein the second chemotherapeutic agent comprises a biologically active complex having anti-tumour activity, wherein the biologically active complex consists of: a peptide of at least 10 amino acids comprising an alpha-helical structure; and oleic acid or an oleate salt, in a ratio of at least 3 oleic acid or oleate salt molecules per peptide molecule. The first chemotherapeutic agent and/or second chemotherapeutic agent of the method of treating or preventing cancer can further comprise any of the features described above for the first, second and third aspects of the invention.

The compositions in accordance with this aspect of invention are suitably pharmaceutical compositions in a form suitable for topical use, for example as creams, ointments, gels, or aqueous or oily solutions or suspensions. These may include the commonly known carriers, fillers and/or expedients, which are pharmaceutically acceptable. Topical solutions or creams suitably contain an emulsifying agent together with a diluent or cream base. The compositions can be delivered by any route. For example, the compositions can be administered orally, particularly for targeting the gastrointestinal tract. The compositions can be administered topically, particularly for targeting mucosal surfaces. Further delivery routes include administration to the circulatory system, for example by intravenous or intramuscular injection. Where the bladder is targeted, the compositions can be administered intravesically, typically by urinary catheter.

Typically, the preparation of a biologically active complex according to the invention may be carried out as described elsewhere (see, for example, WO2018/116156). In brief, preparation can be carried out simply by mixing together a suitable peptide and oleic acid or a salt thereof, for example in a solution such as an aqueous solution. The ratio of oleate:peptide added to the mixture is suitably in the range of from 20:1 to 3:1, for instance in a ratio of oleate:peptide of about 5:1. The mixing can be carried out at a temperature of from 0-50° C., conveniently at ambient temperature and pressure. This simple preparation method provides a particular advantage for the use of such peptides in the complexes. The methods can be carried out in situ, when required for treatment. Thus kits may be provided comprising peptides and salts for mixing immediately prior to administration.

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 shows a schematic of a murine bladder tumor model for determining a dose-dependent therapeutic effect of alpha1-oleate: Bladder cancer was induced in C57BL/6 female mice by intra-vesical instillation of MB49 cells (2×10⁵ in 100 μl PBS). Mice were treated by intravesical instillation of alpha1-oleate (1.7 mM, 8.5 mM or 17 mM) on days 3, 5, 7, 9 and 11 and sacrificed on day 12. Sham treated mice received PBS.

FIG. 2 shows the dose dependent effect of alpha1-oleate, deduced from macroscopic inspection of gross bladder pathology.

FIG. 3 shows a comparison of bladder weight, bladder size and tumor area, data is presented as means±SEMs of two experiments (n=6+5 mice, * P<0.05, ** P<0.01 and *** P<0.001 compared to sham treated mice).

FIG. 4 shows accumulation of alpha1-oleate in tumor tissue: Alexa-Fluor 568-labeled alpha1-oleate was used to challenge tumor-bearing mice and to track the molecule in tumor tissue. (A) Scheme of intravesical Alexa-alpha1-oleate challenge. Intravesical instillations were performed on day 11 and the tissues were harvested after 6 hours. (B) Detection of Alexa-Fluor 568-labeled alpha1-oleate in tumor-bearing mice by confocal imaging of frozen tissue sections. Healthy mice challenged with labeled alpha1-oleate were used as controls. (C) Quantification of fluorescence intensity in (B). Means±SEMs of 5 images/dose.

FIG. 5 shows dose-dependent changes in gene expression in tumor-bearing mice: (A) Heat map showing a dose-dependent reduction in the number of regulated genes in treated mice. Red (left): upregulated genes, blue (right): down-regulated genes, cutoff fold change >2.0 compared to healthy bladder. (B) A dose-dependent reduction in the molecular mechanisms of cancer pathway genes. (C) Individual regulated genes in this pathway. (D) Principal component analysis of mRNA profiles in whole bladder tissue. Increasing doses of alpha1-oleate shifted the transcriptomic profiles from the tumor bearing, sham treated cluster towards the healthy bladder tissue cluster. (E) Scatter plot of probes comparing transcriptomic profiles in bladders from tumor-bearing mice to healthy mice (log₂ signal intensity values). (F) Venn diagram of significantly regulated genes in the sham treated, and alpha1-oleate treated mice compared to healthy bladders.

FIG. 6 shows a schematic of the murine bladder tumor model: bladder cancer was induced in C57BL/6 female mice by intra-vesical instillation of MB49 cells (2×10⁵ in 100 μl PBS). Mice were treated by intravesical instillation of mitomycin C (25 μg; * 25 μg is half of the therapeutic dose in mice, 50 μg i.p. gives rise to systemic toxicity) or alpha1-oleate (8.5 mM) on days 3, 5, 7, 9 and 11 and sacrificed on day 12. Sham treated mice received PBS.

FIG. 7 shows bladder size (and any effect of tumor size on the macroscopic appearance of the bladders) after sacrifice of mice according to the model of FIG. 1, wherein the mice have been treated with sham, 8.5 mM alpha1-oleate or 25 μg mitomycin, healthy controls are also shown (all shown at the same magnification).

FIG. 8 shows the schematic of FIG. 1, modified to investigate the therapeutic effect of the alpha1-oleate and mitomycin C combination.

FIG. 9 shows bladder size (and any effect of tumor size on the macroscopic appearance of the bladders) after sacrifice of mice according to the model of FIG. 3, wherein the mice have been treated with sham, 1.7 mM alpha1-oleate, 25 μg mitomycin and 1.7 mM alpha1-oleate+25 μg mitomycin (all shown at the same magnification).

FIG. 10 shows study on bladder size (and any effect of tumor size on the macroscopic appearance of the bladders) after sacrifice of the 1.7 mM alpha1-oleate+25 μg mitomycin study of FIG. 4 in comparison with the bladder size (and any effect of tumor size on the macroscopic appearance of the bladders) of mice according to the same model but treated with 17 mM alpha1-oleate (all shown at the same magnification).

FIG. 11 shows a schematic of a murine bladder tumor model for determining a therapeutic effect of alpha1-oleate, mitomycin C (MMC) and the combination of the two: Bladder cancer was induced in C57BL/6 female mice by intra-vesical instillation of MB49 cells (2×10⁵ in 100 μl PBS). Mice were treated by intravesical instillation of alpha1-oleate (1.7 mM); MMC (25 μg); and alpha1-oleate (1.7 mM) and MMC (25 μg) on days 3, 5, 7, 9 and 11 and sacrificed on day 12. Sham treated mice received PBS.

FIG. 12 shows the effect of alpha1-oleate, MMC and alpha1-oleate and MMC, deduced from macroscopic inspection of gross bladder pathology.

FIG. 13 shows the effect of alpha1-oleate, MMC and alpha1-oleate and MMC, deduced from inspection of the bladder histology.

FIG. 14 shows a schematic of a murine bladder tumor model for determining a dose-dependent therapeutic effect of alpha1-oleate, mitomycin C (MMC) and the combination of the two: Bladder cancer was induced in C57BL/6 female mice by intra-vesical instillation of MB49 cells (2×10⁵ in 100 μl PBS). Mice were treated by intravesical instillation of alpha1-oleate (1.7 mM and 8.5 mM); MMC (25 μg); and alpha1-oleate (1.7 mM and 8.5 mM) and MMC (25 μg) on days 3, 5, 7, 9 and 11. The Sham group was sacrificed on day 12, treated groups were sacrificed in weeks 4 or 8. Sham treated mice received PBS.

FIG. 15 shows the dose dependent effect of alpha1-oleate, the effect of MMC and the dose dependent effect of alpha1-oleate and MMC, deduced from macroscopic inspection of gross bladder pathology.

FIG. 16 shows the dose dependent effect of alpha1-oleate, the effect of MMC and the dose dependent effect of alpha1-oleate and MMC, deduced from inspection of the bladder histology.

FIG. 17 shows a schematic of a murine bladder tumor model for determining a therapeutic effect of alpha1-oleate; mitomycin C (MMC); epirubicin (Epi); the combination of alpha1-oleate and MMC; and the combination of alpha1-oleate and Epi: Bladder cancer was induced in C57BL/6 female mice by intra-vesical instillation of MB49 cells (2×10⁵ in 100 μl PBS). Mice were treated by intravesical instillation of alpha1-oleate (1.7 mM); MMC (25 ug); Epi (25 ug); alpha1-oleate (1.7 mM) and MMC (25 ug); and alpha1-oleate (1.7 mM) and Epi (25 ug) on days 3, 5, 7, 9 and 11 and sacrificed on day 12. Sham treated mice received PBS.

FIG. 18 shows the effect of alpha1-oleate, Epi, and alpha1-oleate and Epi, deduced from macroscopic inspection of gross bladder pathology.

FIG. 19 shows a schematic similar to that shown in FIG. 17.

FIG. 20 shows the effect of alpha1-oleate, MMC, Epi, alpha1-oleate and MMC, and alpha1-oleate and Epi, deduced from macroscopic inspection of gross bladder pathology.

FIG. 21. Therapeutic efficacy of alpha1-oleate and Mitomycin C in a murine bladder cancer model.

(a) Schematic of the treatment model. Bladder cancer was induced in C57BL/6J female mice by intravesical instillation of MB49 cells (2×10⁵ in 50 μl PBS). The treatment group received alpha1-oleate (1.7 mM or 8.5 mM) or MMC (25 μg/mL) alone or in combination on days 3, 5, 7, 9 and 11. Sham treated mice received PBS and all mice were sacrificed on day 12. (b) Treatment effects deduced from macroscopic inspection of the bladders. (c, d, e) Pathology score, bladder weight, bladder size and tumor area (see also FIG. 20). Data is presented as means±SEMs of two experiments (n=5+6 mice per group, and analyzed by one-way ANOVA).

FIG. 22. Reduction in tumor size by alpha1-oleate and Mitomycin C, alone or in combination.

Tumor areas were compared between sham treated mice and mice receiving 1.7 mM of alpha1-oleate or 25 μg of MMC alone or in combination. (a) Sham treated mice show large tumors filling the bladder lumen (dotted line). (b-c) Alpha1-oleate (1.7 mM) and MMC treated mice show a reduction in tumor size. (d) A further reduction in tumor size in mice treated with a combination of alpha1-oleate (1.7 mM) and MMC (25 μg/mL), (e) Healthy bladders served as negative controls. Representative images are shown (n=6+5 mice per group). (n=5+6 mice per group, and analyzed by one-way ANOVA). The tumor area was identified in H&E-stained whole bladder sections and quantified using ImageJ.

FIG. 23. Therapeutic efficacy of alpha1-oleate and Epirubicin in a murine bladder cancer model.

(a) Schematic of the treatment model. The treatment group received alpha1-oleate by intravesical instillation (alpha1-oleate 1.7 mM or 8.5 mM) or Epirubicin (25 μg/mL) alone or in combination on days 3, 5, 7, 9 and 11. Sham treated mice received PBS and all mice were sacrificed on day 12. (b) Gross bladder pathology defined by macroscopic inspection. (c, d, e) Pathology score, bladder weight, bladder size and tumor area (see also FIG. 22). Data is presented as means±SEMs of two experiments (n=5+5 mice per group, and analyzed by one-way ANOVA).

FIG. 24. Reduction in tumor size by alpha1-oleate and Epirubicin therapy, alone or in combination. (a) Sham treated mice show large tumors filling the bladder lumen (dotted line). (b-c) Alpha1-oleate (1.7 mM or 8.5 mM) and Epirubicin treated mice show a reduction in tumor size. (d, e) Further reduction in tumor size in mice receiving a combination of alpha1-oleate (1.7 mM or 8.5 mM) and Epirubicin. The tumor area was identified in H&E-stained whole bladder sections and quantified using ImageJ.

FIG. 25. Prevention of tumor recurrence by a combination of alpha1-oleate and Mitomycin C.

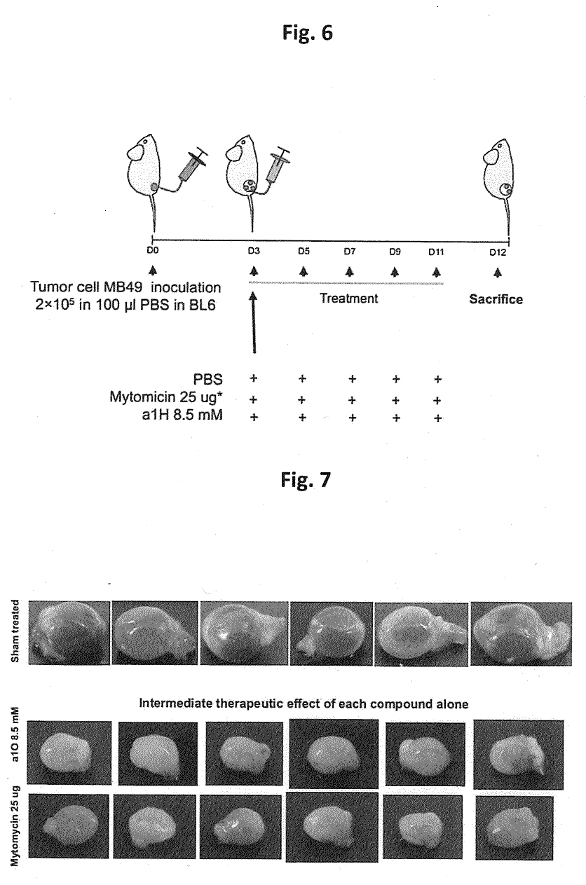

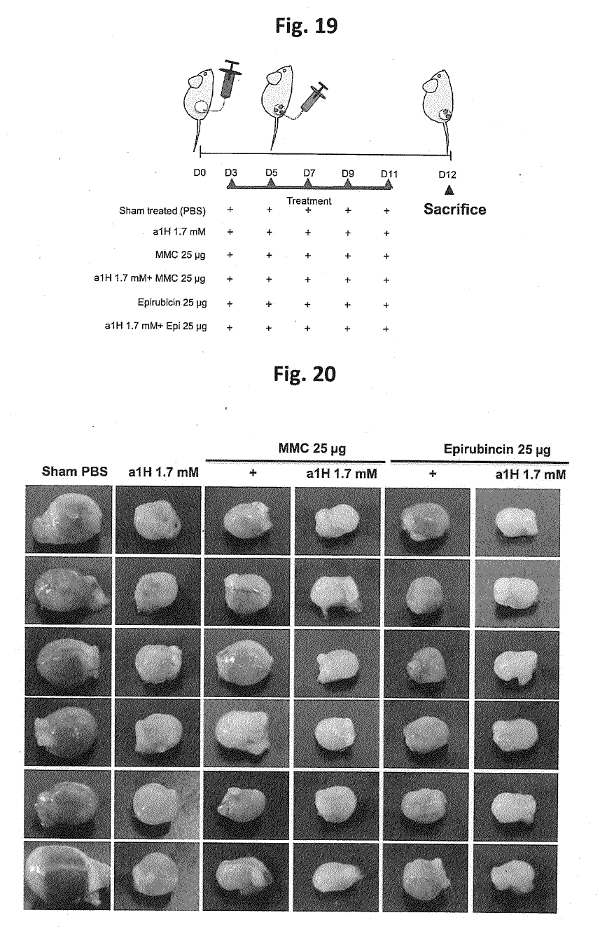

The combination of alpha1 and Mitomycin C showed a prolonged protective effect, preventing tumor relapse for 4 weeks. (a) Effect of alpha1-oleate and MMC alone or in combination, defined by macroscopic inspection of the bladders (See also schematic in FIG. 19a). (c, d, e) Reduced pathology score, bladder weight, bladder size (p<0.001, see also FIG. 6). Data is presented as means±SEMs of two experiments (n=5+5 mice).

FIG. 26. Tumor parameters at long-term follow up of mice treated with alpha1-oleate or Mitomycin C.

(a) Quantification of pathology score, bladder size, bladder weight and tumor areas. (b-e) Tumor areas were compared after 4 weeks between mice receiving 1.7 mM and 8 mM of alpha1-oleate and 25 μg of MMC. (b, c) Evidence of tumor recurrence in mice receiving alpha1-oleate (1.7 mM) and MMC (25 μg) alone. (d-e) Prevention of tumor recurrence by the combination of alpha1-oleate 1.7 mM or 8.5 mM with Mitomycin C prevented tumor relapse. Tumors were not detected in H&E-stained whole bladder sections. Representative images are shown (n=5+5 mice per group).

FIG. 27. Tumor parameters at long-term follow up of mice treated with alpha1-oleate or Epirubicin.

(a) Quantification of pathology score, bladder size, bladder weight and tumor areas. (b-e) Tumor areas were compared after 4 weeks between mice receiving 1.7 mM and 8 mM of alpha1-oleate and 25 μg of Epirubicin. (b, c) Evidence of tumor recurrence in mice receiving alpha1-oleate (1.7 mM) and Epirubicin (25 μg) alone. (d-e) Prevention of tumor recurrence by the combination of alpha1-oleate 1.7 mM or 8.5 mM with Epirubicin prevented tumor relapse. Tumors were not detected in H&E-stained whole bladder sections. Representative images are shown (n=5+5 mice per group).

### DETAILED DESCRIPTION

The present invention provides a novel molecular solution to targeting and killing tumor cells with greater efficacy and precision using a combination therapy. The inventors have shown that the effect of a biologically active complex according to the invention upon tumor cells is greater than just the direct anti-tumor properties of the complex. The complex also renders the tumor cells more susceptible to the effect of other chemotherapeutic agents. It is further noted that complexes according to the invention do not appear to affect healthy cells (see Example 2), suggesting that any enhanced effects of the further chemotherapeutic agents are limited to tumour cells. This enhancement may, for example, occur through disruption of the cell wall (such as by direct disruption of the lipid bilayer or through disruption of membrane-bound proteins, including ion channels) and/or through modulation of gene expression, particularly genes involved in the molecular mechanisms of the cancer pathway (see Example 3). In any event, the data collected for complexes according to the invention and other chemotherapeutic agents show a clear synergistic enhancement in therapeutic effect when comparing use individually (Example 4) and use in combination (Example 5).

### Example 1—Dose Escalation Study of Alpha1-Oleate in Tumor-Bearing Mice

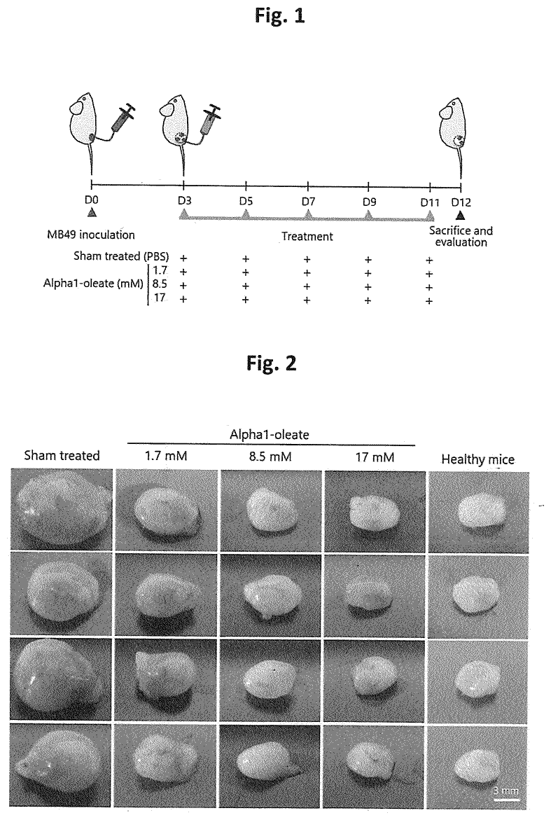

We performed a dose escalation study in the murine MB49 bladder cancer model (inoculation at day 0, FIG. 1). Mice in the treatment group received five intravesical instillations of alpha1-oleate on days 3, 5, 7, 9 and 11 and sham treated mice had PBS instilled at these time points. The sham treated mice developed palpable tumors that altered the macroscopic appearance of the bladders, compared to controls not receiving tumor cells. The tumors were growing rapidly, from the mucosa and the tumor mass gradually filled the bladder lumen, replacing functional bladder tissue.

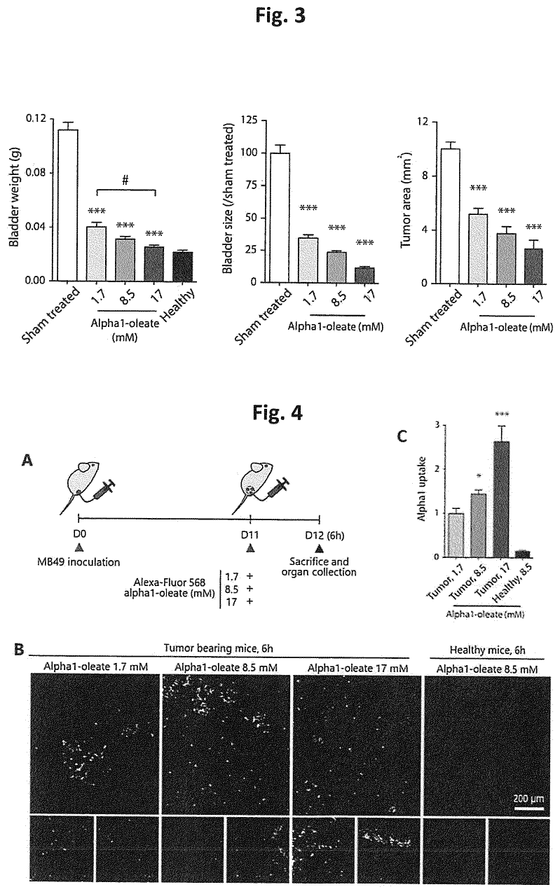

Treated mice were administered increasing concentrations of alpha1-oleate (1.7, 8.5 or 17 mM in 100 μl, 5-6 mice per group, 2 experiments per dose). Bladders were harvested on day 12 and evaluated macroscopically (FIG. 2), bladder size, weight and tumor area were recorded (FIG. 3). Tumor growth was attenuated after treatment with 1.7 mM of alpha1-oleate, with a reduction in bladder weight, bladder size and tumor size (P<0.001 compared to sham treated mice, FIG. 3). A further, dose-dependent reduction in bladder weight, bladder size and tumor size was recorded in mice receiving 8.5 mM of alpha1-oleate (P<0.001) and at the highest concentration (17 mM) no macroscopically visible tumor tissue remained. The bladder size and weight in these mice did not differ from that in healthy control mice.

The results identify alpha1-oleate as a tumoricidal complex with therapeutic efficacy that increases with increasing doses of the compound, until most of the tumor is cleared.

### Example 2—Accumulation of Alpha1-Oleate in Tumor Tissue

To examine if alpha1-oleate reaches tumor tissue, tumor-bearing mice were inoculated with Alexa-Fluor 568 labeled alpha1-oleate (day 11, n=2). Bladders were harvested six hours after instillation and frozen tissue sections were subjected to confocal imaging. The Alexa-Fluor labeled complex was shown to accumulate in tumor tissue, in a dose-dependent manner (1.7 versus 8.5 mM, 8.5 mM versus 17 mM) (FIGS. 4A-C) but Alexa-Fluor 568 alpha1-oleate was not detected in healthy mice subjected to the same procedure (FIGS. 4B and C). The results were confirmed by dot blot analysis of tumor-bearing mice treated with alpha1-oleate, using anti-alpha1 antibodies. The accumulation of alpha1-oleate in tumor tissue was confirmed by staining of frozen tissue sections with anti-alpha1 antibodies. Tissues from healthy mice exposed to alpha1-oleate were negative.

### Example 3—Dose-Dependent Inhibition of Gene Expression in Tumor-Bearing Mice

To further characterize the tumor response to alpha1-oleate, total bladder RNA was subjected to whole-genome transcriptomic profiling. Gene expression was compared between the sham treated group and mice receiving increasing doses of alpha1-oleate. Healthy mice served as controls.

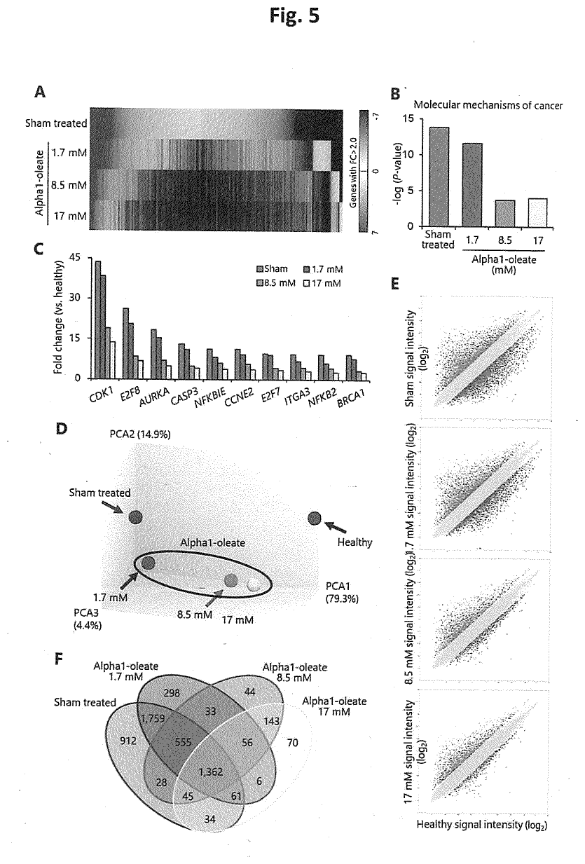

The transcriptomic data revealed major differences in gene expression between the sham treated and the alpha1-oleate treated mice. A dose-dependent reduction in the number of differentially expressed genes was observed, consistent with the drastic reduction in tumor size in the treated mice (fold change compared to healthy, see heat map in FIG. 5A). The molecular mechanisms of cancer pathway and comprising genes were deactivated in a dose-dependent manner, as shown by Ingenuity Pathway Analysis (IPA) (FIGS. 5B and C). By principal component analysis (PCA, FIG. 5D), the tumor-bearing, sham treated mice formed a distinct transcriptomic profile, which was clearly separated from the treated mice and the healthy controls. PCA1 (79.3% of variation) was dominated by differences between sham treated and healthy bladders. With increasing doses, the treated mice shifted towards the healthy expression profile. PCA2 (14.9% of variation) distinguished the untreated group from the alpha1-oleate treated mice. The conclusions were supported by scatter plot analysis of probe signal intensities (FIG. 5E), revealing a dose-dependent decrease in the number of differentially expressed transcripts between healthy control and treated mice, also illustrated in the Venn diagram (FIG. 5F).

### Example 4—Examination of Individual Therapeutic Effect of Alpha1-Oleate and Mitomycin C

We performed another study in the murine MB49 bladder cancer model where mice in the treatment group received five intravesical instillations of 8.5 mM alpha1-oleate or 25 μg mitomycin C on days 3, 5, 7, 9 and 11 and sham treated mice had PBS instilled at these time points (FIG. 6).

Bladders were harvested on day 12 and evaluated (FIG. 7). Tumor growth was attenuated to a similar extent after treatment with 8.5 mM alpha1-oleate or 25 μg mitomycin, with a reduction in bladder weight, bladder size and tumor size compared to sham treated mice.

### Example 5—Examination of Combined Therapeutic Effect of Alpha1-Oleate and Mitomycin

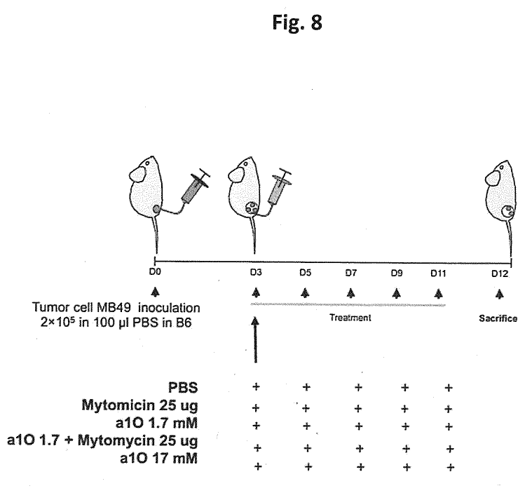

A modified version of the previous study was performed, wherein mice in the treatment group received five intravesical instillations of 1.7 mM alpha1-oleate, 25 μg mitomycin, or the combination of 1.7 mM alpha1-oleate+25 μg mitomycin and sham treated mice had PBS instilled at these time points (FIG. 8).

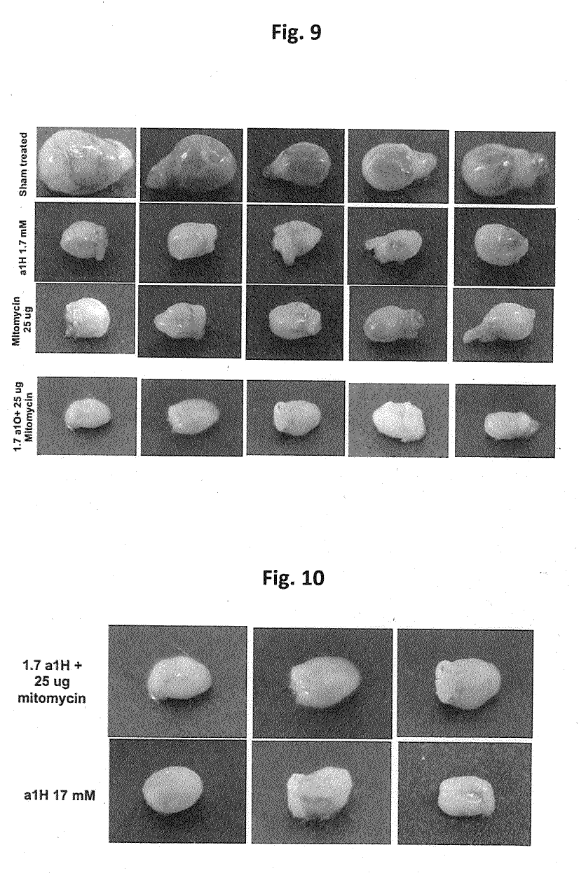

The results show that treatment with the combination of 1.7 mM alpha1-oleate+25 μg mitomycin produces bladders that are equivalent or almost equivalent to healthy bladders, unlike treatment with either the 1.7 mM alpha1-oleate or 25 μg mitomycin alone (FIG. 9). Furthermore, FIG. 10 shows that treatment with the combination is producing an effect on the tumors that appears equivalent with alpha1-oleate alone at 10 times the concentration (i.e. 17 mM alpha1-oleate).

### Example 6—Examination of Combined Therapeutic Effect of Alpha1-Oleate and Mitomycin

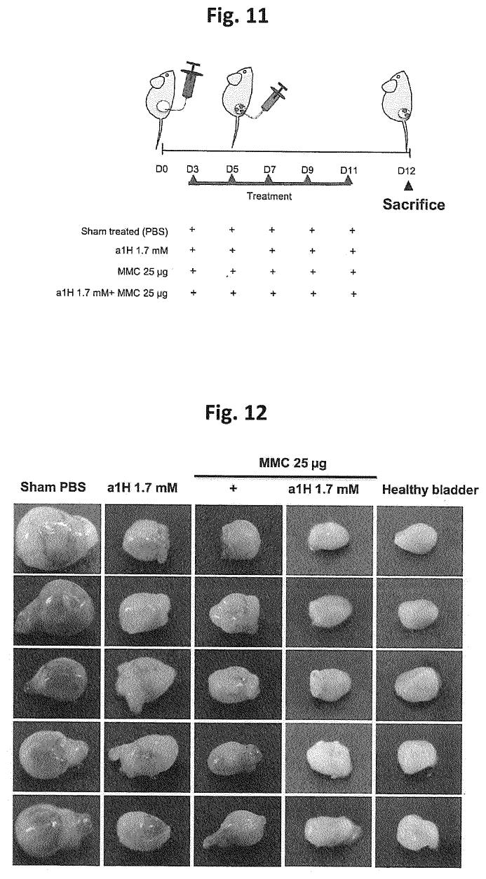

A modified version of the previous study was performed, wherein mice in the treatment group received five intravesical instillations of 1.7 mM alpha1-oleate, 25 μg mitomycin, or the combination of 1.7 mM alpha1-oleate+25 μg mitomycin and sham treated mice had PBS instilled at these time points (FIG. 11).

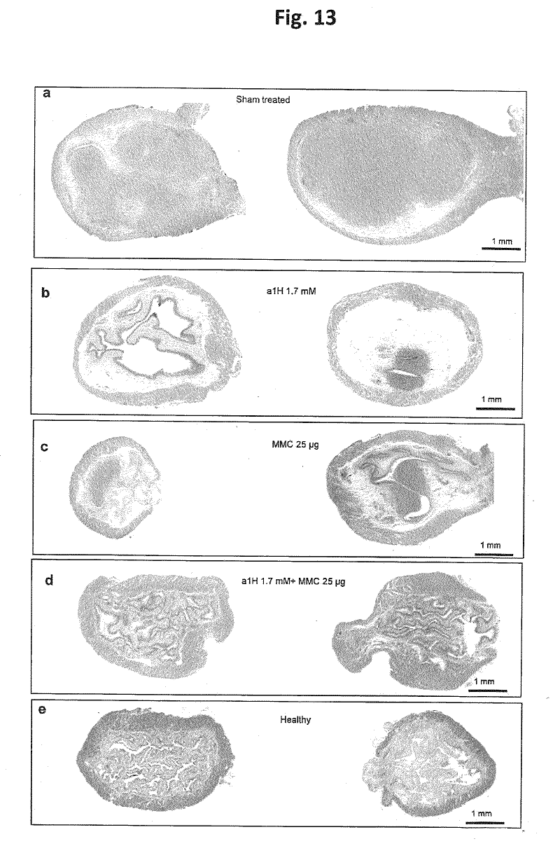

The results show that treatment with the combination of 1.7 mM alpha1-oleate+25 μg mitomycin produces bladders that are equivalent or almost equivalent to healthy bladders, unlike treatment with either the 1.7 mM alpha1-oleate or 25 μg mitomycin alone (FIG. 12). Furthermore, FIG. 13 shows that treatment with the combination produces bladder histology that is equivalent to or almost equivalent to that seen in healthy bladders.

### Example 7—Examination of Combined Therapeutic Effect of Alpha1-Oleate and Mitomycin

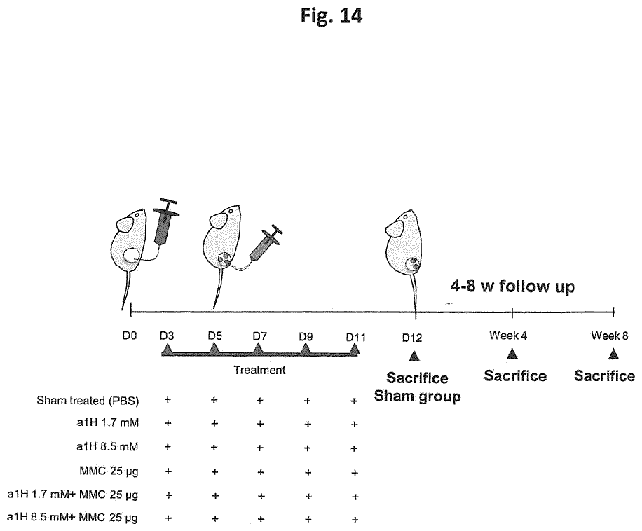

A modified version of the previous study was performed, wherein mice in the treatment group received five intravesical instillations of 1.7 or 8.5 mM alpha1-oleate, 25 μg mitomycin, or the combination of 1.7 or 8.5 mM alpha1-oleate+25 μg mitomycin and sham treated mice had PBS instilled at these time points (FIG. 14).

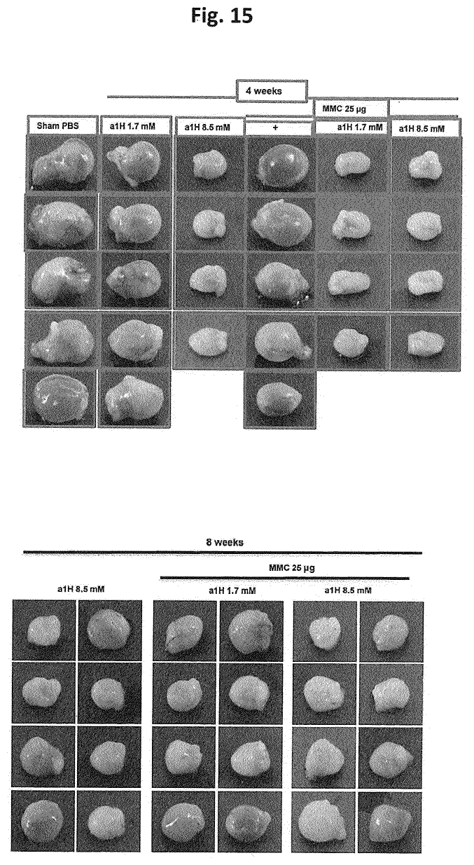

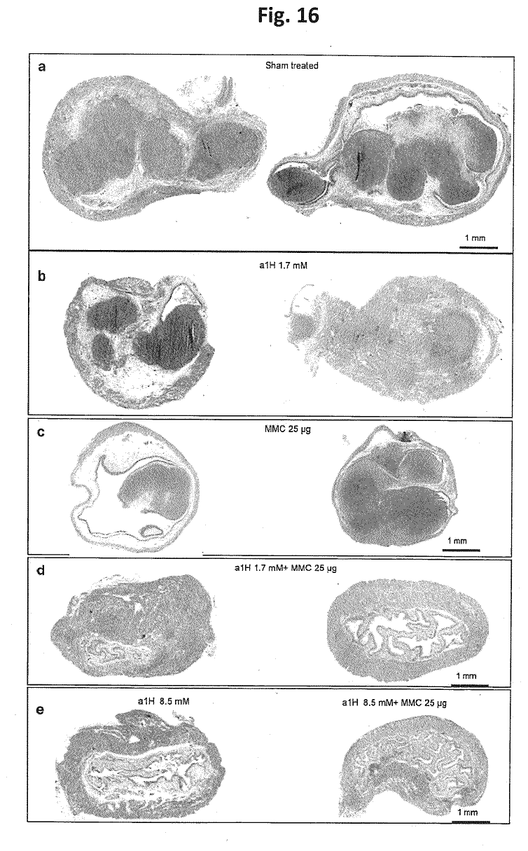

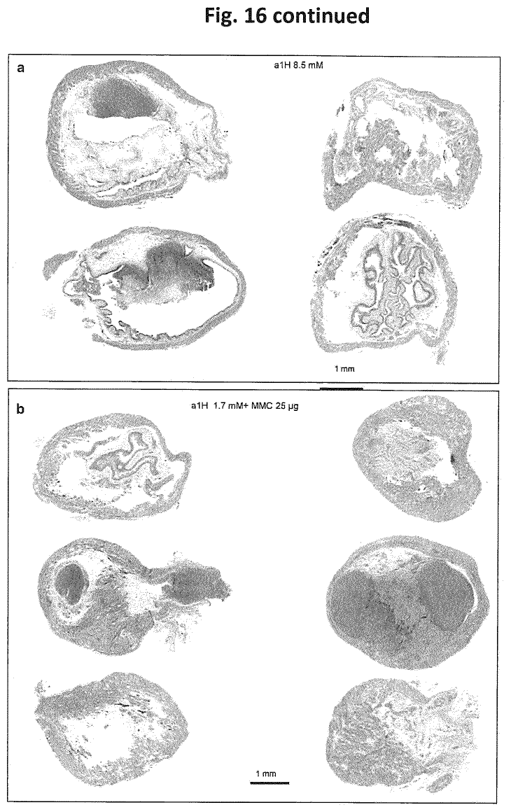

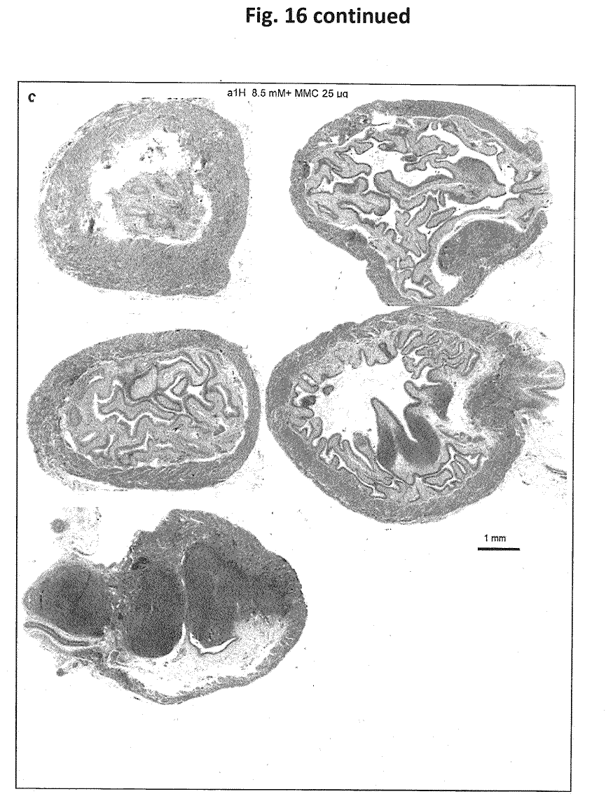

The results show that treatment with the combination of 1.7 mM alpha1-oleate+25 μg mitomycin produces bladders that are equivalent or almost equivalent to healthy bladders, unlike treatment with either the 1.7 mM alpha1-oleate or 25 μg mitomycin alone (FIG. 15). Furthermore, FIG. 15 shows that treatment with the combination is producing an effect on the tumors that appears equivalent with alpha1-oleate alone at 5 times the concentration (i.e. 8.5 mM alpha1-oleate). FIG. 16 shows that treatment with the combination produces bladder histology that is equivalent to or almost equivalent to that seen in healthy bladders. FIGS. 15 and 16 also show that the effects are maintained over extended time periods of 4 and 8 weeks from inoculation.

### Example 8—Examination of Combined Therapeutic Effect of Alpha1-Oleate and Mitomycin or Epirubicin

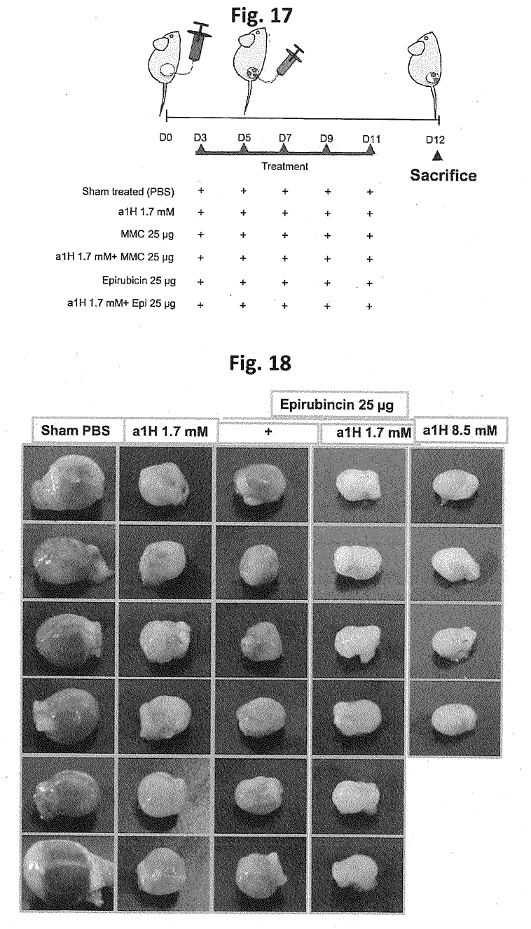

A modified version of the previous study was performed, wherein mice in the treatment group received five intravesical instillations of 1.7 mM alpha1-oleate, 25 μg mitomycin, 25 μg epirubicin, the combination of 1.7 or mM alpha1-oleate+25 μg mitomycin or the combination of 1.7 or mM alpha1-oleate+25 μg epirubicin and sham treated mice had PBS instilled at these time points (FIG. 17).

The results show that treatment with the combination of 1.7 mM alpha1-oleate+25 μg epirubicin produces bladders that are equivalent or almost equivalent to those from mice treated with the combination of 1.7 mM alpha1-oleate+25 μg mitomycin and to healthy bladders, unlike treatment with either the 1.7 mM alpha1-oleate, 25 μg mitomycin or 25 μg epirubicin alone (FIG. 18). Furthermore, FIG. 18 shows that treatment with the combination of alpha1-oleate and epirubicin is producing an effect on the tumors that appears equivalent with alpha1-oleate alone at 5 times the concentration (i.e. 8.5 mM alpha1-oleate).

Materials and Methods

Chemicals and Antibodies

Oleic acid (Croda, batch number: 0001120439), poly-L-lysine solution (Sigma, Cat #RNBF4239), Alexa-Fluor 568 protein labeling kit (Thermo Scientific, Cat #A10238), ECL Plus detection reagent (GE healthcare, Cat #RPN2132), Richard-Allan Scientific Signature Series Hematoxylin and Eosin-Y (Thermo Scientific, Cat #7211 and 7111), DAPI (Sigma, Cat #D9542), anti-alpha lactalbumin (Mybiosource, Cat #MBS175270), monoclonal mouse anti-β-actin (Sigma-Aldrich, Cat #A2228), polyclonal rabbit anti-mouse IgG-HRP (Dako, Cat #P0260), polyclonal goat anti-rabbit IgG-HRP (Cell Signaling, Cat #7074), rabbit polyclonal anti-VEGF (Abcam, Cat #ab46154), mouse monoclonal anti-Ki-67 (BD Biosciences, Cat #556003), rabbit monoclonal anti-Cyclin D1 (Thermo Fisher, Cat #SC8396), goat anti-rabbit IgG Alexa-Fluor 488 (Thermo Fisher, Cat #A-11034), goat anti-mouse IgG Alexa 568 (Thermo Fisher, Cat #A-11004), DRAQ5 (Abcam, Cat #ab108410).

Peptide Synthesis and Complex Generation

Alpha1 was synthesized using Fmoc solid phase chemistry (Mimotopes). Alpha1 stock concentration was diluted in PBS and mixed with five times concentration of oleic acid in PBS to achieve the final alpha1-oleate complex. The alpha1 sequence is: Ac-KQFTKAELSQ LLKDIDGYGG IALPELIATM FHTSGYDTQ-OH.

Bladder Cancer Model

C57BL/6 female mice were bred at the Department of Laboratory Medicine, Lund University and used at ages from 7 to 12 weeks. For procedures, mice were anesthetized by intraperitoneal injection of a cocktail of ketamine (1.48 mg in 100 μl of NaCl, Intervet) and xylazine (0.22 mg in 100 μl of NaCl, Vetmedic). On day 0, the bladder was emptied and preconditioned by intravesical instillation of 100 μl poly-L-lysine solution (0.1 mg/ml) through a soft polyethylene catheter (Clay Adams) with an outer diameter of 0.61 mm. After 30 minutes, MB49 mouse bladder carcinoma cells (2×10⁵ in 100 μl PBS) were instilled. On days 3, 5, 7, 9 and 11, 100 μl of alpha1-oleate (alpha1: 1.7 mM, 8.5 mM or 17 mM, oleic acid: 8.5 mM, 42.5 mM or 85 mM, respectively) or PBS (sham treated controls) were instilled. Mice remained under anesthesia on preheated blocks with the catheter in place to prolong tumor exposure to the peptide-oleate complexes (approximately 1 hour). Groups of 5-6 mice for each treatment and control were sacrificed after 12 days, and bladders were imaged and processed for histology. Two independent experiments were performed.

Statistical Analysis

Results are presented as means±SEMs and groups are compared by one-way ANOVA. P values were calculated by Student's t test and one way analysis of variance followed by Bonferroni's post hoc testing using GraphPad Prism version 7 (GraphPad Software Inc.). P<0.05 was considered statistically significant. * P<0.05; ** P<0.01; *** P<0.001.

### Example 9—Further Examination of Combination Therapies

Materials and Methods

Chemicals and Antibodies

Oleic acid (Croda, batch number: 0001120439), poly-L-lysine solution (Sigma, Cat #RNBF4239), Richard-Allan Scientific Signature Series Hematoxylin and Eosin-Y (Thermo Scientific, Cat #7211 and 7111), Epirubicin (Sigma-Aldrich, Cat #E9406) and Mitomycin Sigma-Aldrich, Cat #M0440).

Peptide Synthesis and Complex Generation

We have identified the N-terminal, alpha-helical domain of alpha-lactalbumin as a tumoricidal entity, which forms a complex with oleic acid (Ho J, Rydstrom A, Manimekalai MSS, Svanborg C, Grüber G. Low resolution solution structure of HAMLET and the importance of its alpha-domains in tumoricidal activity. PLoS One 2012; 7: e53051). For this study, we synthesized the 39 amino acid peptide (aa 1-39, Ac-KQFTKAELSQLLKDIDGYGGIALPELIATMFHTSGYDTQ-OH), using Fmoc solid phase chemistry with the purity of >95% (Polypeptide group, France). To form the alpha1-oleate complex, the alpha1 peptide was mixed with sodium oleate at a 1:5 molar ratio. The stock solution was further diluted in PBS to the appropriate concentration for each experiment.

Bladder Cancer Model

MB49 (RRID:CVCL_7076) cells were provided by Sara Mangsbo, Uppsala University, Sweden. MB49 bladder cancer was established as previously described (Mossberg A-K, Hou Y, Svensson M, Holmqvist B, Svanborg C. HAMLET treatment delays bladder cancer development. The Journal of urology 2010; 183: 1590-7). C57BL/6 female mice were bred at the Department of Laboratory Medicine, Lund University and used at ages from 7 to 12 weeks. For procedures, mice were anesthetized by intraperitoneal injection of a cocktail of ketamine (1.48 mg in 100 μl of 0.9% NaCl solution, Intervet) and xylazine (0.22 mg in 100 μl of 0.9% NaCl solution, Vetmedic). On day 0, the bladder was emptied and preconditioned by intravesical instillation of 100 μl of poly-L-lysine solution (0.1 mg/ml) through a soft polyethylene catheter (Clay Adams) with an outer diameter of 0.61 mm. After 30 minutes, MB49 mouse bladder carcinoma cells (2×10⁵ cells in 50 μl media) were instilled. On days 3, 5, 7, 9 and 11, mice were randomly assigned and instilled to the alpha1-oleate (1.7 mM or 8.5 mM), chemotherapeutic drugs (MMC 25 μg, epirubicin 25 μg), combination of alpha1-oleate with chemotherapeutic agent or sham PBS treatment group. The catheter was left in place for about one minute, but as the mice remained under anesthesia, the time to voiding (dwelling time) of the substance was 2-3 hours. Groups of 5-6 mice for each treatment and control were sacrificed at day 12. Follow up groups of 5-8 mice were sacrificed at week 4 and week 8. Bladders were imaged and processed for histology or RNA extraction. Two independent experiments were performed for each condition. All experiments were performed with mycoplasma-free cells.

Histology

Bladders were embedded in O.C.T. compound (VWR), and successive 5 μm sections were collected from the center of each bladder and placed on positively-charged microscope slides (Superfrost/Plus; Thermo Fisher Scientific). For hematoxylin-eosin (H&E) staining, Richard-Allan Scientific Signature Series Hematoxylin 7211 was used followed by Eosin-Y 7111 for counterstaining. Images was captured using the AX10 microscope (Carl Zeiss). The tumor circumferences were measured for analysis of the tumor area using ImageJ software.

Gene Expression Analysis

Frozen bladder tissue was pulverized using liquid nitrogen. (Tran T. Hien1, Ambite, I., Lam Yim Wan, Butler, D., Tran T. Hiep, Höglund, U., Babjuk, M. and Svanborg, C. Bladder cancer treatment without toxicity—A dose-escalation study of alpha1-oleate. International Journal of Cancer. in press). Total RNA was extracted (RNeasy Mini kit, Qiagen). 100 ng of total RNA was amplified using the GeneChip 3′IVT Express Kit and then fragmented. Next, labeled aRNA was hybridized onto Mouse Genome 430 PM array strips (Affymetrix) for 16 hours at 45° C., washed, stained (Applied Biosystems, ThermoFisher Scientific) and scanned using the GeneAtlas system (Affymetrix). All samples passed the internal quality controls included in the array strips (signal intensity by signal to noise ratio; hybridization and labeling controls; sample quality by GAPDH signal and 3′-5′ ratio <3). Transcriptomic data was normalized using Robust Multi Average implemented in the Transcriptome Analysis Console software (v.4.0.1.36, Applied Biosystems, ThermoFisher Scientific). Fold change was calculated by comparing tumor-bearing bladders or treated healthy bladders to untreated healthy bladder control tissue. Relative expression levels were analyzed and genes with an absolute fold change >2.0 were considered as differentially expressed. Heat-maps were constructed using the Gitools 2.1.1 software. Differentially expressed genes were functionally characterized using the Ingenuity Pathway Analysis (IPA, Qiagen) software.

Statistical Analysis

Results are presented as means±SEMs. p values were calculated by Student's t test or one-way ANOVA followed by Bonferroni's post hoc testing using Prism version 7 (GraphPad Software Inc.). p<0.05 was considered statistically significant. * p<0.05; ** p<0.01; *** p<0.001.

Results

Effects of Combination Therapy with Alpha1-Oleate and Mitomycin

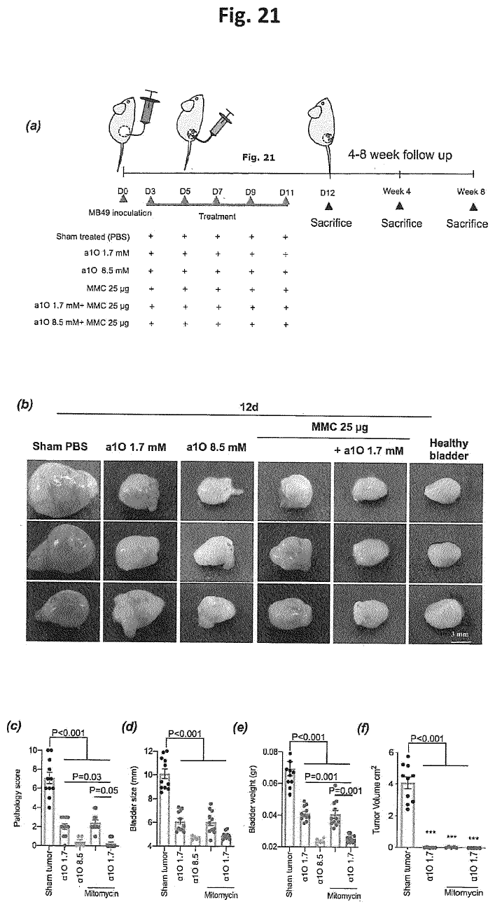

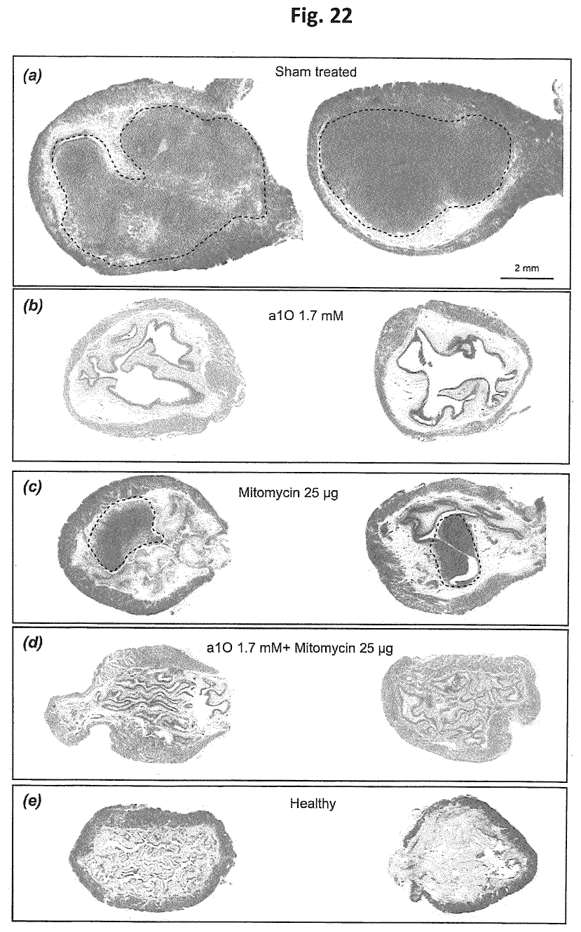

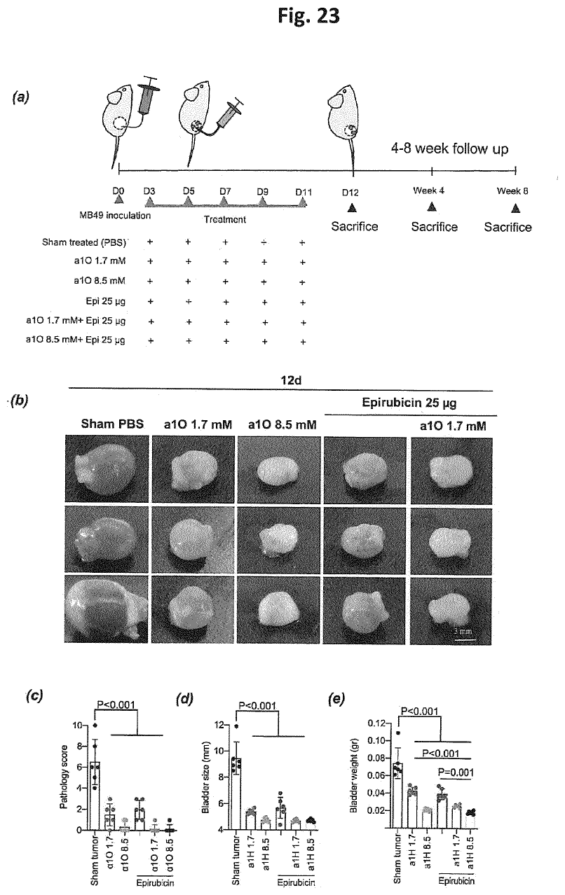

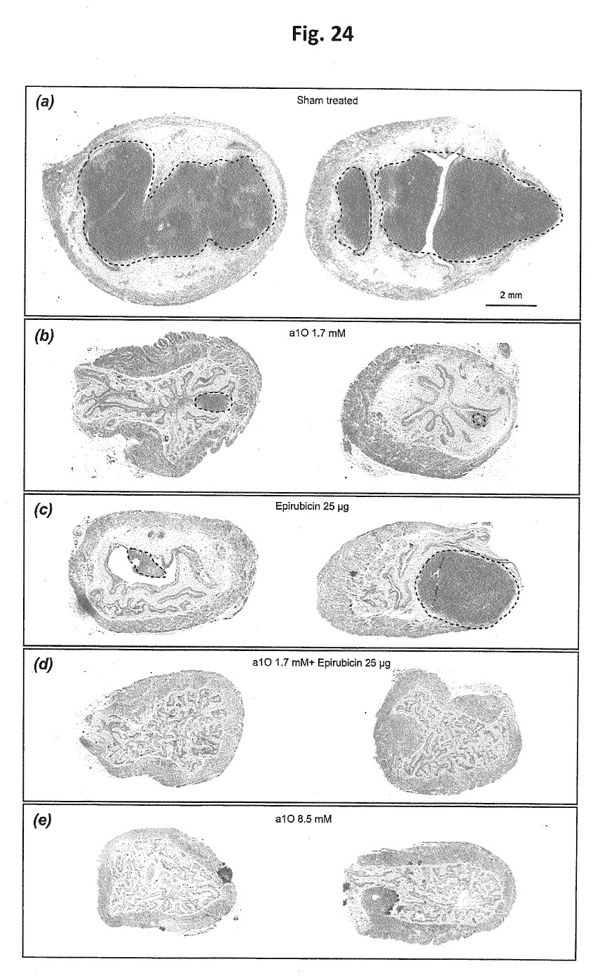

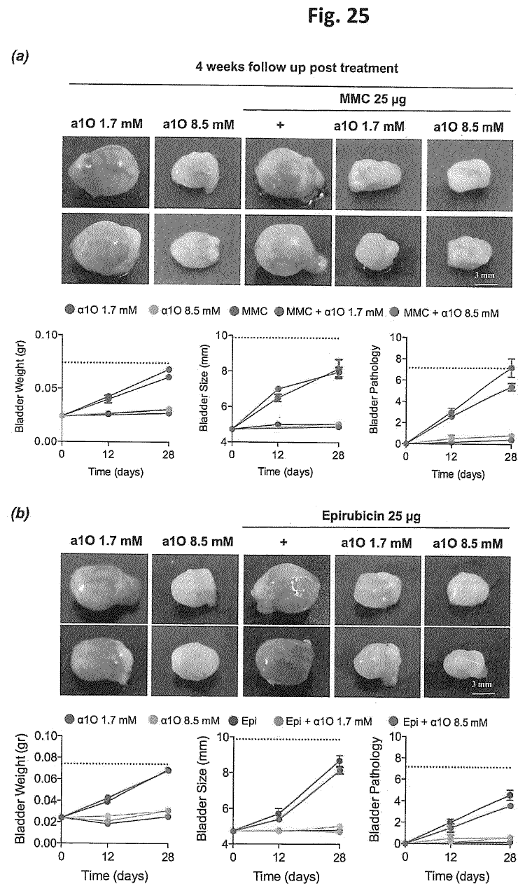

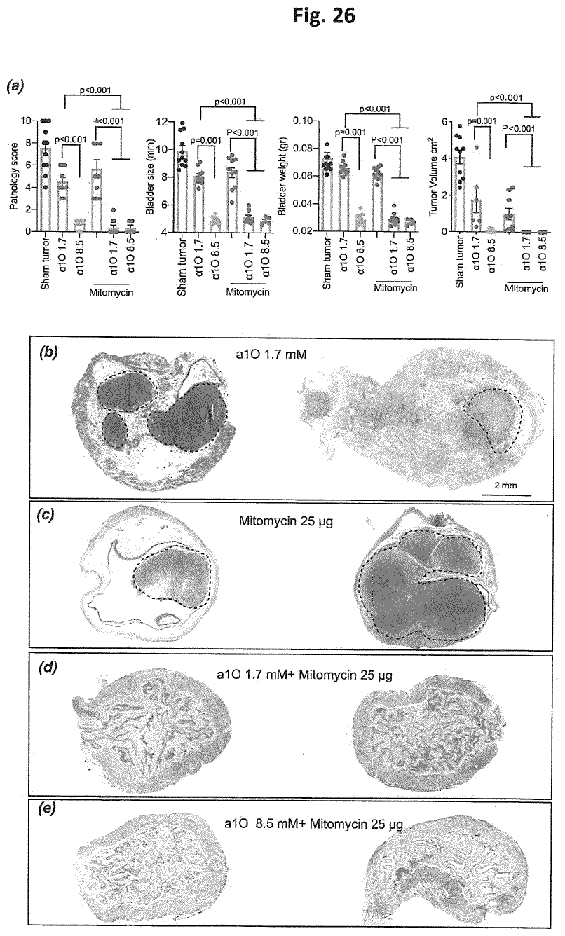

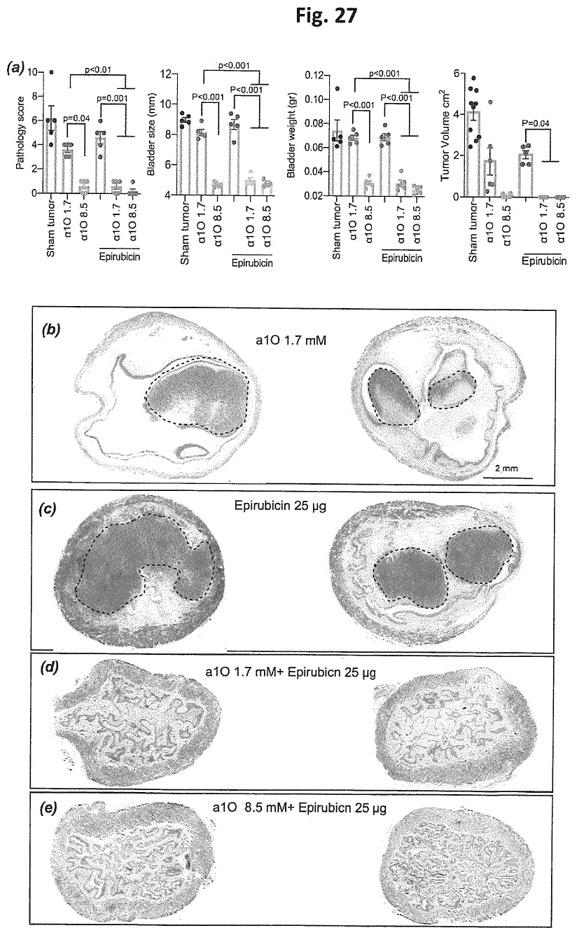

The effects of alpha1-oleate and Mitomycin C were investigated in the murine MB49 bladder cancer model (for study design see FIG. 19A). Bladder cancer was established by intra-vesical instillation of MB49 cells and treatment was initiated on day 3. The treatment group received alpha1-oleate (1.7 mM or 8.5 mM), MMC (0.1 mL, 25 ug/dose) or a combination of alpha1-oleate (1.7 mM or 8.5 mM) with MMC (0.1 mL, 25 ug/dose). Instillations were performed on days 3, 5, 7, 9 and 11 and sham treated mice received PBS. Bladders were harvested at sacrifice on day 12 and tumor development was quantified as bladder weight, bladder size, tumor size and pathology score (FIG. 21c-f). The tumor area was quantified in H&E stained, whole-bladder tissue sections (FIG. 22).

The sham treated mice developed large tumors that filled the bladder lumen after 12 days (12/12 mice, FIG. 21b). Therapeutic efficacy of alpha1-oleate and Mitomycin C was demonstrated to the sham-treated group. Tumor progression was delayed by alpha1-oleate alone in a dose-dependent manner (p<0.01, FIG. 19c-f) and by Mitomycin C alone (0.1 mL, 25 ug/dose), (FIGS. 21 and 22).

The combined effect of Mitomycin and alpha1-oleate was examined by first inoculating the mice with alpha1-oleate (1.7 or 8.5 uM). After 2 hours, the mice received a second inoculation of Mitomycin (see FIG. 21a) and the procedure was repeated once daily on day 3, 5, 7, 9 and 11 followed by sacrifice on day 12. A strong synergistic effect was detected between the chemotherapeutic agents and alpha1-oleate (FIG. 21b). The therapeutic effect was further increased in mice receiving a combination of alpha1-oleate and Mitomycin C (p=0.03), most prominently in mice receiving the higher dose or alpha1-oleate (8.5 mM). No tumor tissue was detected in the treated mice by visual inspection at sacrifice and the bladder weight and bladder size did not differ from that in healthy bladders from control mice (p=0.99, FIGS. 21c and 21e). The reduction in tumor area was confirmed by microscopy after H&E staining. No tumor was detected in any of the treatment groups (p<0.001) (FIG. 20). As alpha1-oleate at a dose of 8.5 mM was efficient, a further increase of the therapeutic effect of MMC combination therapy was not detected at 12 days (p=0.99).

Effects of Combination Therapy with Alpha1-Oleate and Epirubicin

The mechanism of action of Epirubicin resembles that of Mitomycin, affecting DNA intercalation and arresting cellular growth. The two compounds also show a similar toxicity profile. Subsequent experiments therefore addressed the therapeutic efficacy of alpha1-oleate and Epirubicin alone and in combination (FIG. 23a). The combined effect of Epirubicin and alpha1-oleate was examined by first inoculating the mice with alpha1-oleate (1.7 or 8.5 uM). After 2 hours, the mice received a second inoculation of Epirubicin (see FIG. 23a) and the procedure was repeated once daily on day 3, 5, 7, 9 and 11 followed by sacrifice on day 12.

Tumor progression was delayed by Epirubicin (p<0.001 compared to sham treated controls), as defined by bladder weight, bladder size, tumor size and pathology score (FIG. 22c-f). The reduction in tumor area was confirmed by H&E staining of whole-bladder tissue sections. The therapeutic effect of Epirubicin alone was comparable to that of alpha1-oleate (1.7 mM, p=0.97). At the higher dose (8.5 mM), alpha1-oleate was more efficient (FIG. 24, p=0.004).

A strong synergistic effect was detected between Epirubicin and alpha1-oleate. No tumor tissue was detected in the treated mice by visual inspection at sacrifice and the bladder weight and bladder size did not differ from that in healthy bladders from control mice (FIG. 23c-e and FIG. 21c-e). The reduction in tumor area was confirmed by H&E staining, where no tumor tissue was detected (FIG. 24). A dose of 8.5 mM was more efficient than 1.7 mM (p=0.001) as stand-alone therapy but did not significantly increase the therapeutic effect of the MMC combination therapy, after 12 days (p=0.62).

The results suggest that alpha1-oleate treatment enhances the effects of the chemotherapeutic agents Mitomycin and Epirubicin, to the point of preventing tumor development.

Long-Term Follow Up

The duration of the therapeutic effect was evaluated by following the mice for a total of four weeks (FIG. 28). The sham treated group was not followed beyond 12 days, when they were sacrificed, due to the rapid growth of the tumor. Extended protection was observed in the treatment groups, however. After 4 weeks, mice receiving low dose of alpha1-oleate (1.7 mM), MMC or Epirubicin showed an increase in tumor growth. In the combination therapy groups however, mice remained protected with no evidence of tumor relapse (FIGS. 25 and 26). The combined effect of low dose alpha1-oleate (1.7 mM) and MMC or Epirubicin (25 uG/dose) was stronger than that of MMC or Epirubicin alone (p<0.001 compared to MMC or Epirubicin) but comparable to the higher dose of alpha1-oleate showing no significant differences (8.5 uM). Further protection was observed in mice treated with the combination of 8.5 mM of alpha1-oleate and Epirubicin (FIG. 27).

1. A first chemotherapeutic agent and a second chemotherapeutic agent for use in a combination cancer therapy, wherein the second chemotherapeutic agent comprises a biologically active complex having anti-tumour activity, wherein the biologically active complex consists of:
a peptide of at least 10 amino acids comprising an alpha-helical structure; and
oleic acid or an oleate salt, in a ratio of at least 3 oleic acid or oleate salt molecules per peptide molecule.

2. A first chemotherapeutic agent for use in treating cancer; wherein the agent is for administration to a subject to which a second chemotherapeutic agent will be administered, or has been administered, or is being administered; and wherein the second chemotherapeutic agent comprises a biologically active complex having anti-tumour activity, wherein the biologically active complex consists of:
a peptide of at least 10 amino acids comprising an alpha-helical structure; and
oleic acid or an oleate salt, in a ratio of at least 3 oleic acid or oleate salt molecules per peptide molecule.

3. A second chemotherapeutic agent for use in for use in treating cancer; wherein the agent is for administration to a subject to which a first chemotherapeutic agent will be administered, or has been administered, or is being administered; and wherein the second chemotherapeutic agent, wherein the second chemotherapeutic agent comprises a biologically active complex having anti-tumour activity, wherein the biologically active complex consists of:
a peptide of at least 10 amino acids comprising an alpha-helical structure; and
oleic acid or an oleate salt, in a ratio of at least 3 oleic acid or oleate salt molecules per peptide molecule.

4. A first chemotherapeutic agent and/or second chemotherapeutic agent for use according to any preceding claim, wherein the first chemotherapeutic agent is an intravesical chemotherapeutic agent, a topical chemotherapeutic agent, a DNA-interactive chemotherapeutic agent, a DNA-alkylator chemotherapeutic agent, and/or a DNA-crosslinker chemotherapeutic agent.

5. A first chemotherapeutic agent and/or second chemotherapeutic agent for use according to any preceding claim, wherein the first chemotherapeutic agent is selected from the group consisting of: atezolizumab, avelumab, Bacille Calmette-Guerin, bevacizumab, carbozantinib, cephalexin, ciprofloxacin, cisplatin, doxorubicin hydrochloride, durvalumab, eflornithine, epirubicin, erdafitinib, erlotinib, fenretinide, gemcitabine, gefitinib, lapatinib, mitomycin C, nivolumab, pazobanib, pembrolizumab, rapamycin, selenium, sorafenib, thiotepa, urocidin, valrubicin, and vicinium, preferably thiotepa, mitomycin C, Bacille Calmette-Guerin or epirubicin, more preferably mitomycin C or epirubicin.

6. A first chemotherapeutic agent and/or second chemotherapeutic agent for use according to any preceding claim, wherein the peptide of the biologically active complex is of a protein which has membrane perturbing activity.

7. A first chemotherapeutic agent and/or second chemotherapeutic agent for use according to any preceding claim, wherein the peptide of the biologically active complex, where present in the complex, has an increased conformational fluidity of three-dimensional structure as compared to the non-complexed peptide as indicated by an increased peak width on at least some ¹H NMR peaks of the complex as compared to the corresponding width of the peaks of a ¹H NMR of the non-complexed peptide.

8. A first chemotherapeutic agent and/or second chemotherapeutic agent for use according to any preceding claim, wherein the peptide of the biologically active complex lacks cysteine residues.

9. A first chemotherapeutic agent and/or second chemotherapeutic agent for use according to any preceding claim, wherein the peptide of the biologically active complex consists of up to 50 amino acids in length.

10. A first chemotherapeutic agent and/or second chemotherapeutic agent for use according to any preceding claim, wherein the peptide of the biologically active complex is alpha-lactalbumin or SAR-1, or a variant or fragment thereof, preferably an N-terminal fragment thereof.

11. A first chemotherapeutic agent and/or second chemotherapeutic agent for use according to any preceding claim, wherein the peptide of the biologically active complex comprises or consists of any of SEQ ID NOs: 1-4, or variants or fragments thereof.
(SEQ ID NO: 1)
KQFTKAELSQ LLKDIDGYGG IALPELIATM FHTSGYDTQ
(SEQ ID NO: 2)
MAGWDIFGWF RDVLASLGLW NKH
(SEQ ID NO 3)
KQFTKAELSQ LLKDI
(SEQ ID NO 4)
MAGWDIFGWF RDVLA.

12. A first chemotherapeutic agent and/or second chemotherapeutic agent for use according to any preceding claim, wherein the peptide of the biologically active complex consists of any of SEQ ID NOs: 1-4, preferably SEQ ID NO: 1.

13. A first chemotherapeutic agent and/or second chemotherapeutic agent for use according to any preceding claim, wherein the use is treatment or prevention of carcinomas, lymphomas or brain tumours, preferably treatment or prevention of cancer of the GI tract, mucosal cancer, bladder cancer, kidney cancer, lung cancer, glioblastomas and skin papilloma, more preferably treatment or prevention of bladder cancer.

14. A pharmaceutical composition comprising a first chemotherapeutic agent, a second chemotherapeutic agent, and a pharmaceutically acceptable carrier, excipient and/or adjuvant, wherein the second chemotherapeutic agent comprises a biologically active complex having anti-tumour activity, wherein the biologically active complex consists of:
a peptide of at least 10 amino acids comprising an alpha-helical structure; and
oleic acid or an oleate salt, in a ratio of at least 3 oleic acid or oleate salt molecules per peptide molecule.

15. A pharmaceutical composition according to claim 14 for use in cancer therapy.

16. A pharmaceutical composition according to claim 14 or 15, wherein the first chemotherapeutic agent is mitomycin, preferably mitomycin C.

17. A pharmaceutical composition according to claim 14 or 15, wherein the first chemotherapeutic agent is epirubicin.

18. A pharmaceutical composition according to any of claims 14 to 17, wherein the second chemotherapeutic agent comprises a biologically active complex having anti-tumour activity, wherein the biologically active complex consists of:
a peptide having or comprising a peptide the sequence:
KQFTKAELSQ LLKDIDGYGG IALPELIATM FHTSGYDTQ;
and
oleic acid or an oleate salt, in a ratio of at least 3 oleic acid or oleate salt molecules per peptide molecule.

19. A method of treating or preventing cancer, the method comprising administration to a subject in need thereof of a first chemotherapeutic agent in conjunction with a second chemotherapeutic agent, wherein the second chemotherapeutic agent comprises a biologically active complex having anti-tumour activity, wherein the biologically active complex consists of:
a peptide of at least 10 amino acids comprising an alpha-helical structure; and
oleic acid or an oleate salt, in a ratio of at least 3 oleic acid or oleate salt molecules per peptide molecule.
